(19) United States
(12) Patent Application Publication
(10) Pub. No.: US 2010/0003346 A1
(43) Pub. Date: Jan. 7, 2010

(54) COMBINATION METHODS OF TREATING CANCER

(76) Inventors: Eliezer Flescher, Hod Hasharon (IL); Alina Heyfets, Tel Aviv (IL); Max Herzberg, Sitrya (IL)

Correspondence Address: FENNEMORE CRAIG, 3003 NORTH CENTRAL AVENUE, SUITE 2600, PHOENIX, AZ, 85012, US

(21) Appl. No.: 12/307,557

(22) PCT Filed: Jul. 10, 2007
(86) PCT No.: PCT/IL07/00863

§ 371 Date: Feb. 4, 2009

Related U.S. Application Data
(60) Provisional application No. 60/819,375, filed on Jul. 10, 2006.

(51) Int. Cl. A61K 33/24; A61K 31/235; A61K 31/47; A61K 31/335; A61K 31/351; A61K 31/70; A61K 31/175; A61P 35/04

(52) U.S. Cl. 424/649; 514/532; 514/311; 514/449; 514/459; 514/23; 514/589

(57) ABSTRACT

The present invention relates to compositions and methods for treating cancer, by administering a combination comprising a jasmonate derivative (e.g., methyl jasmonate or a compound of any of formulae I through VII or any of the jasmonate derivatives exemplified by such formulae) and at least one other agent selected from a chemotherapeutic agent (e.g., a nitroso-urea, a platinum compound, a taxane derivative, an antitumor antibiotic) and an inhibitor of glycolysis (e.g., 2-deoxy-D-glucose). The jasmonate derivative and the at least one other agent together provide a therapeutic effect, which is preferably synergistic (cooperative).

### FIELD OF THE INVENTION

The present invention relates to the treatment of cancer using combination therapy comprising a jasmonate derivative in combination with a chemotherapeutic agent and/or an inhibitor of glycolysis.

### BACKGROUND OF THE INVENTION

Jasmonates are a family of plant stress hormones, derived from linolenic acid by the octadecanoid pathway, which are found in minute quantities in many edible plants. Stress hormones such as the jasmonate family have evolved in plants, and are released in such times of stress such as extreme UV radiation, osmotic shock, heat shock and pathogen attack, to initiate various cascades which end in appropriate responses. Examples of members of the jasmonate family are jasmonic acid, which is crucial to intracellular signaling in response to injury, and methyl jasmonate (MJ), which causes induction of a proteinase inhibitor, that accumulates at low concentrations in response to wounding or pathogenic attacks. Use of jasmonates for the treatment of mammalian cancer has been disclosed in U.S. Pat. No. 6,469,061, the contents of which are incorporated by reference in their entirety. In U.S. Pat. No. 6,469,061, it was shown that jasmonates were directly cytotoxic for various types of human cancer cells derived from breast, prostate, skin and blood cancers. MJ in particular was shown to be effective in preventing development of lymphomas in mice (see, U.S. Pat. No. 6,469,061 and Fingrut, O. and E. Flescher. 2002 (1)). MJ was also shown to induce death in human leukemia, prostate, breast and melanoma cell lines, as well as in leukemic cells from chronic lymphocytic leukemia (CLL) patients (2,3).

While jasmonates elicited death in human leukemic Molt-4 cells, they do not damage normal peripheral blood erythrocytes (4), normal lymphocytes (2) and human sperm cells. See also WO 02/080890, the contents of which are incorporated by reference in their entirety. These results strongly support the conclusion that jasmonates specifically target transformed cells.

PCT International Patent Publication WO 2005/054172, the contents of which are incorporated by reference herein in their entirety, discloses novel halogenated jasmonate derivatives, pharmaceutical compositions comprising the derivatives, and their use for reducing cancer cell growth and for treating cancer.

International Patent Publications WO 2007/066336 and WO 2007/066337, the contents of each of which are incorporated by reference herein in their entirety, disclose novel jasmonate derivatives, pharmaceutical compositions comprising the derivatives, and their use for reducing cancer cell growth and for treating cancer.

It has further been shown that jasmonates are capable of inducing both necrotic and apoptotic death in Molt-4 human lymphoblastic leukemia cells (1). Furthermore, jasmonates are capable of killing cancer cells in a manner independent of cellular mRNA transcription, protein translation (5), and p53 expression (6).

Recent studies have analyzed the mechanism through which jasmonates induce cell death. Mitochondria were found to play a pivotal role in the mechanism of action of jasmonates. Indeed, jasmonates act directly on mitochondria, resulting in cell death (2). Jasmonates induced mitochondrial membrane depolarization and cytochrome c release in intact cancer cells (2). More importantly, MJ induced swelling and cytochrome c release in mitochondria isolated from human leukemia and hepatoma cell lines, as well as leukemic cells from CLL patients (2). However, jasmonates did not induce cytochrome c release or swelling in mitochondria isolated from normal lymphocytes. It thus appears that the difference between the normal and cancer cells exists at the mitochondrial level. Interestingly, jasmonates did not induce swelling in mitochondria isolated from immortal, but non-transformed, 3T3 human fibroblasts (2), suggesting that neoplastic transformation renders the mitochondria susceptible to jasmonates. Thus, MJ has direct mitochondriotoxic effects, strongly suggesting that mitochondria are target organelles of jasmonates. In support of this contention, inhibitors of the opening of the mitochondrial permeability transition pore complex (PTPC, a pore mediating mitochondrial perturbation resulting in cell death) reduced significantly the toxic effects of MJ on cancer cells and on mitochondria isolated from these cells. These studies (2) show that jasmonates kill cancer cells in a PTPC-dependent manner. The direct effect of jasmonates on mitochondria should endow them with the ability to bypass pre-mitochondrial anti-apoptotic mutations, thereby making this class of anti-cancer agents potentially active against a variety of drug-resistant tumors.

In accordance with principles for selecting agents for use in combination chemotherapy regimens, drugs with different mechanisms of action and with additive or synergistic cytotoxic effects on the tumor can be combined (7). Multi-agent therapy has three important theoretical advantages over single-agent therapy. First, it can maximize cell kill while minimizing host toxicities by using agents with non-overlapping dose-limiting toxicities. Second, it may increase the range of drug activity against tumor cells with endogenous resistance to specific types of therapy. Finally, it may also prevent or slow the development of newly resistant tumor cells (7). Virtually, almost all curative chemotherapy regimens for cancer employ multi-agent drug combinations (8). Although ideal drug combinations would be those that are synergistically active against malignant cells without increased systemic toxicity, additive anti-tumor activity with favorable toxicity profile can also be clinically beneficial (9).

Traditional chemotherapeutic agents can be classified by mechanism of action. The alkylating agents impair cell function by forming covalent bonds with the amino, carboxyl, sulfhydryl, and phosphate groups in biologically important molecules. The most important sites of alkylation are DNA, RNA, and proteins. Alkylating agents depend on cell proliferation for activity but are not cell-cycle-phase-specific. Alkylating agents are classified according to their chemical structures and mechanisms of covalent bonding; this drug class includes the nitrogen mustards, nitroso-ureas (BCNU) and platinum complexes (cisplatin) (7). Taxanes are semisynthetic derivatives of extracted precursors from the needles of yew plants. These drugs have a novel 14-member ring, the taxane. Unlike the vinca alkaloids, which cause microtubular disassembly, the taxanes (e.g., taxol) promote microtubular assembly and stability, therefore blocking the cell cycle in mitosis (7). Antitumor antibiotics like adriamycin intercalate DNA at guanine-cytosine and guanine-thymine sequences, resulting in spontaneous oxidation and formation of free oxygen radicals that cause strand breakage (7).

Recently, it has been shown using three in vitro models of simulated hypoxia (10-12), that cells under hypoxic conditions are more sensitive than cells under aerobic conditions to agents that inhibit glycolysis, such as 2-deoxy-D-glucose (2DG). Because a slowly proliferating tumor population can be selectively killed with glycolytic inhibitors, combining such agents with chemotherapeutic drugs, which target the rapidly dividing aerobic cells, should raise the overall efficacies of these treatments (10-12). Indeed, the combination of 2DG and cisplatin is more effective than either agent alone when applied to various cell lines that are rapidly proliferating in vitro (13). Similar in vitro synergism has been observed with the combination of 2DG and adriamycin (ADR) in MCF7 cells (14). It has recently been found that 2DG and MJ had an additive effect on ATP depletion in B-lymphoma cells expressing either wt or mutant p53 (6). The basis for this additive effect is probably the inhibitory actions jasmonates and 2DG have on different cellular pathways generating ATP, oxidative phosphorylation and glycolysis, respectively.

BCL1 (B-cell leukemia/lymphoma 1) is a spontaneous murine leukemia originally described in 1978 by Slavin and Strober in a 2-year-old female BALB/cKa (H-2d) mouse (15). The tumor bearing mouse has high leukocyte counts and marked splenomegaly. The cytological features of the BCL1 cells are essentially the same as those seen in human disorders of well-differentiated lymphocytic lymphoma and chronic lymphocyte leukemia (CLL) (16). Thus, they provide a useful animal model for the study of these diseases. With regard to CLL, although many patients have a benign disease and live a normal life span, others have a more malignant form of disease and have very shortened life span after diagnosis due to resistance to chemotherapy (17). Chemotherapeutic drugs, such as chlorambucil, prednisone, and certain monoclonal antibodies directed to specific cell surface proteins induce B-CLL apoptosis in vivo, although complete remission is difficult to attain and all patients eventually relapse (18). Purine analogues induce significant clinical improvement but are associated inevitably with immune suppression, resulting in opportunistic infections (19). In addition, the combination of 9-β-D-arabinofuranosyl-2-fluoroadenine (fludarabine) and cyclophosphamide induces myelosuppression (20). It is therefore important to search for new agents which may be useful as novel therapies for CLL, alone or in combination with already known drugs.

The pharmacological activity of jasmonate compounds makes them attractive candidates as therapeutic agents for the treatment of cancer, alone or in combination with additional chemotherapeutic agents.

### SUMMARY OF THE INVENTION

The present invention relates to compositions and methods for treating cancer, by administering a combination comprising a jasmonate derivative (e.g., methyl jasmonate or a compounds of any of formulae I through VII or any of the jasmonate derivatives exemplified by such formulae) in combination with at least one other agent selected from a chemotherapeutic agent (e.g., a nitroso-urea, a platinum compound, a taxane derivative, an antitumor antibiotic), an inhibitor of glycolysis (e.g., 2-deoxy-D-glucose) or combinations thereof. The jasmonate derivative and the at least one other agent collectively are administered in an amount which provides a therapeutic effect, which is preferably synergistic.

It has been unexpectedly discovered that the combination of a first treatment that includes administration of a jasmonate derivative, as described herein, and a second treatment using one or more agents selected from a chemotherapeutic drug and an inhibitor of glycolysis, as described herein, can provide therapeutically effective anticancer effects. In some embodiments, the effect is synergistic, i.e., the jasmonate derivative and the at least one other agent together produce a significantly better anticancer result (e.g., cell growth arrest, apoptosis, induction of differentiation, cell death, etc.) than the additive effects achieved by each individual constituent when administered alone at a therapeutic dose. Preferably, the overall effect of the combined therapy after a course of treatment will be significantly better than the effects achieved with a course of each of the therapeutic agents individually.

The combination of therapy is particularly advantageous, since the dosage of each agent in a combination therapy can be reduced as compared to monotherapy with each agent, while still achieving an overall anti-tumor effect. In addition, due to the synergistic effect, the total amount of drugs administered to a patient can advantageously be reduced, which may result in decreased side effects.

As exemplified herein, the applicants of the present invention investigated the interaction between methyl jasmonate (MJ) and conventional chemotherapeutic agents (e.g., the nitrosourea BCNU, cisplatin, taxol and adriamycin), as well as an inhibitor of glycolysis, 2-deoxy-D-glucose (2DG). MJ was found to act synergistically with several cytotoxic drugs and 2DG in a variety of cell lines. Specifically, MJ exhibited a synergistic effect with taxol in mammary adenocareinoma, lung carcinoma, breast adenocarcinoma and prostate adenocarcinoma cell lines; with cisplatin in pancreatic carcinoma and prostate adenocarcinoma cell lines; with adriamycin in a B-cell leukemia cell line, and with BCNU in a pancreatic carcinoma and B-cell leukemia cell lines. Moreover, in vivo results demonstrate that combined treatment of MJ with adriamycin significantly increased survival of BCL1 leukemia-bearing mice, while MJ or adriamycin alone did not induce increased survival. In addition, MJ was found to act synergistically with 2DG in colon carcinoma, lung carcinoma and breast adenocarcinoma cell lines. The unexpected results underline the importance of the combination of MJ with chemotherapeutic drugs and suggest that it may have clinical value for the treatment of several types of cancer.

The present invention thus relates to a method for treating cancer in a subject in need thereof, comprising administering to the subject a jasmonate derivative in combination with at least one other agent selected from a chemotherapeutic agent and an inhibitor of glycolysis, wherein the jasmonate derivative and the at least one other agent together provide a synergistic therapeutic effect.

In another embodiment, the present invention relates to a method for inhibiting cancer cell proliferation, comprising contacting cancer cells with a jasmonate derivative in combination with at least one other agent selected from a chemotherapeutic agent and an inhibitor of glycolysis, wherein the jasmonate derivative and the at least one other agent together provide a synergistic effect.

In yet another embodiment, the present invention relates to the use of a jasmonate derivative in combination with at least one other agent selected from a chemotherapeutic agent and an inhibitor of glycolysis, wherein the jasmonate derivative and the at least one other agent together provide a synergistic therapeutic effect.

The term “in combination” or “combined treatment” as used herein denotes any form of concurrent or parallel treatment with at least two distinct therapeutic agents. This term is intended to encompass both concomitant administration of the two treatment modalities, i.e., using substantially the same treatment schedule, as well as overlapping administration in sequential or alternating schedules of each treatment.

The jasmonate derivative and the at least one other chemotherapeutic agent can be administered simultaneously (in the same or in separate dosage forms), or they can be administered sequentially, in any order. The administration can also take place according to alternating dosing schedules, e.g., jasmonate derivative followed by chemotherapeutic agent, then an additional dose of jasmonate derivative, followed by a glycolysis inhibitor, etc. All administration schedules, including simultaneous, sequential and alternating, are contemplated by the present invention.

In one currently preferred embodiment, the jasmonate derivative is methyl jasmonate. In another currently preferred embodiment, the jasmonate derivative is a compound represented by the formula:

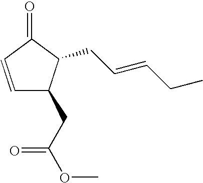

In another currently preferred embodiment, the jasmonate derivative is a compound represented by the formula:

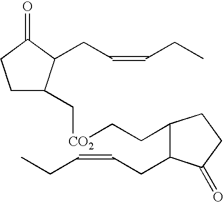

In another currently preferred embodiment, the jasmonate is a compound of formula 9:

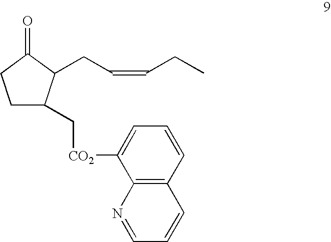

In other embodiments, however, the jasmonate derivative can be jasmonic acid or any derivative thereof. Suitable jasmonate derivatives are disclosed in U.S. Pat. No. 6,469,061, PCT International Patent Application Publication Nos. WO 02/080890, WO 2005/054172, and in WO 2007/066336 and WO 2007/066337. The contents of each of the aforementioned references are incorporated by reference herein in their entirety as if fully set forth herein.

Suitable chemotherapeutic agents include, but are not limited to, alkylating agents, antibiotic agents, antimetabolic agents, hormonal agents, plant-derived agents and their synthetic derivatives, anti-angiogenic agents, differentiation inducing agents, cell growth arrest inducing agents, apoptosis inducing agents, cytotoxic agents, agents affecting cell bioenergetics i.e., affecting cellular ATP levels and molecules/activities regulating these levels, biologic agents, e.g., monoclonal antibodies, kinase inhibitors and inhibitors of growth factors and their receptors, gene therapy agents, cell therapy, e.g., stem cells, or any combination thereof.

In some currently preferred embodiments, the chemotherapeutic agent is a nitroso-urea (e.g., 1,3-bis[2-chloroethyl]-10-nitroso-urea (BCNU)), a platinum compound (e.g., cisplatin), a taxane derivative (e.g., taxol), an antitumor antibiotic (e.g., adriamycin), or any combination thereof. In another currently preferred embodiment, the inhibitor of glycolysis is 2-deoxy-D-glucose (2DG).

In another embodiment, the present invention relates to a method for treating cancer in a subject in need thereof, comprising administering to the subject a jasmonate derivative in combination with at least one other agent selected from the group consisting of a nitroso-urea (e.g., 1,3-bis[2-chloroethyl]-10-nitroso-urea (BCNU), a platinum compound (e.g., cisplatin), a taxane derivative (e.g., taxol), an antitumor antibiotic (e.g., adriamycin), an inhibitor of glycolysis (e.g., 2DG), or any combination thereof, wherein the jasmonate derivative and the at least one other agent together provide a therapeutic effect. In a preferred embodiment, the therapeutic effect is synergistic.

In embodiment, the cancer is lung carcinoma, the jasmonate derivative is methyl jasmonate, and the at least one other agent is taxol. In another embodiment, the cancer is pancreatic carcinoma, the jasmonate derivative is methyl jasmonate, and the at least one other agent is cisplatin or BCNU. In another embodiment, the cancer is breast adenocarcinoma, the jasmonate derivative is methyl jasmonate, and the at least one other agent is taxol. In another embodiment, the cancer is prostate adenocarcinoma, the jasmonate derivative is methyl jasmonate, and the at least one other agent is cisplatin or taxol. In another embodiment, the cancer is B-cell leukemia, the jasmonate derivative is methyl jasmonate, and the at least one other agent is adriamycin or BCNU. In yet another embodiment, the cancer is colon carcinoma, lung carcinoma or breast adenocarcinoma, the jasmonate derivative is methyl jasmonate, and the at least one other agent is 2DG.

The present invention also contemplates pharmaceutical compositions that include a first amount of a jasmonate derivative in combination with a second amount of at least one other agent selected from a chemotherapeutic agent and an inhibitor of glycolysis. The collective amount of jasmonate derivative and at least one other agent provides a synergistic therapeutic anti-cancer effect.

The pharmaceutical compositions of the present invention can be provided in any form known in the art, for example in a form suitable for oral administration (e.g., a solution, a suspension, a syrup, an emulsion, a dispersion, a suspension, a tablet, a pill, a capsule, a pellet, granules and a powder), for parenteral administration (e.g., intravenous, intramuscular, intra-arterial, transdermal, subcutaneous or intraperitoneal), for topical administration (e.g., an ointment, a gel, a cream), for administration by inhalation or for administration via suppository. In one particular embodiment, the active ingredient is dissolved in any acceptable lipid carrier.

The combinations of the present invention are active against a wide range of cancers. The combinations of the present invention are active against a wide range of cancers, including carcinomas, sarcomas, myelomas, leukemias, lymphomas and mixed type tumors. Particular categories of tumors amenable to treatment include lymphoproliferative disorders, breast cancer, ovarian cancer, prostate cancer, cervical cancer, endometrial cancer, bone cancer, liver cancer, stomach cancer, colon cancer, pancreatic cancer, cancer of the thyroid, head and neck cancer, cancer of the central nervous system, cancer of the peripheral nervous system, skin cancer, kidney cancer, as well as metastases of all the above. Particular types of tumors amenable to treatment include: hepatocellular carcinoma, hematoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, Ewing's tumor, leiomyosarcoma, rhabdotheliosarcoma, invasive ductal carcinoma, papillary adenocarcinoma, melanoma, squamous cell carcinoma, basal cell carcinoma, adenocarcinoma (well differentiated, moderately differentiated, poorly differentiated or undifferentiated), renal cell carcinoma, hypernephroma, hypernephroid adenocarcinoma, bile duct carcinoma, choriocarcinoma, seminoma, embryonal carcinoma, Wilms' tumor, testicular tumor, lung carcinoma including small cell, non-small and large cell lung carcinoma, bladder carcinoma, glioma, astrocyoma, medulloblastoma, craniopharyngioma, ependymoma, pinealoma, retinoblastoma, neuroblastoma, colon carcinoma, rectal carcinoma, hematopoietic malignancies including all types of leukemia and lymphoma including: acute myelogenous leukemia, acute myelocytic leukemia, acute lymphocytic leukemia, chronic myelogenous leukemia, chronic lymphocytic leukemia, mast cell leukemia, multiple myeloma, myeloid lymphoma, Hodgkin's lymphoma, non-Hodgkin's lymphoma.

In particular the combinations of the present invention are active against breast cancer, kidney cancer, stomach cancer, leukemia, including lymphoblastic leukemia, lung carcinoma, melanoma and colon cancer.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1: Cytotoxic effect of MJ towards tumor cells lines. Cytotoxicity is calculated as % of control untreated cells, mean±s.e of triplicates. FIG. 1A: CT26, DA-3 and D122. FIG. 1B: TRAMP C1, MIA PaCa-2, MCF7 and BCL1.

FIG. 2: Cytotoxic effect of combined treatments with MJ and chemotherapeutic drugs on different carcinoma cell lines in vitro. Cytotoxicity is calculated as % of control untreated cells, mean±s.e of triplicates. The expected values are assuming additivity. The observed effect values are those actually generated by the combinations of MJ and the cytotoxic drugs.

FIG. 2A. Mia PaCa-2 cells were incubated in the presence of cisplatin or BCNU at the indicated concentrations and/or 1 mM MJ. pV<0.05 comparing expected cytotoxicity for BCNU+MJ (at all indicated concentrations) and cisplatin+MJ (at 1 and 2.5 μg/ml) versus observed effect.

FIG. 2B. MCF7 cells were incubated in the presence of taxol at the indicated concentrations and/or 1 mM MJ. pV<0.05 comparing expected cytotoxicity for taxol+MJ at 2.5 μg/ml versus observed effect.

FIG. 2C. DA-3 cells were incubated in the presence of taxol at the indicated concentrations and/or 0.5 mM MJ. pV<0.05 comparing expected cytotoxicity for taxol+MJ at all indicated concentrations except for 10 μg/ml, versus observed effect.

FIG. 2D. D122 cells were incubated in the presence of taxol at the indicated concentrations and/or 1 mM MJ. pV<0.05 comparing expected cytotoxicity for taxol+MJ at all indicated concentrations versus observed effect.

FIG. 2E. TRAMP C1 cells were incubated in the presence of taxol or cisplatin at the indicated concentrations and/or 0.5 mM MJ (in the case of cisplatin) and 1 mM MJ (in the case of taxol). pV<0.05 comparing expected cytotoxicity for cisplatin+MJ at 2.5 μg/ml versus observed effect. pV<0.05 comparing expected cytotoxicity for taxol+MJ at all indicated concentrations versus observed effect.

FIG. 3: Cytotoxic effect of combined treatments with MJ and chemotherapeutic drugs on BCL1 cells. The cells were pre-incubated with BCNU (A) or adriamycin (B) at the indicated concentrations for 1 h, and MJ at 0.1 mM was added for 24 h. The expected values are assuming additivity. The observed effect values are those actually generated by the combinations of MJ and the cytotoxic drugs. Cytotoxicity is calculated as % of control untreated cells, mean±s.e of triplicates. pV<0.05 comparing expected cytotoxicity for BCNU+MJ at 2.5, 5, 10 μg/ml or adriamycin+MJ at 5, 10, 25 ng/ml versus observed effect.

FIG. 4: Combination of adriamycin (ADR) and MJ, i.v., exhibits a cooperative effect against BCL1 leukemia in vivo. The mice were treated with MJ 60 mg/kg i.v. every day (5 days a week) for 4 weeks. Adriamycin (ADR) was administered i.p. twice, 4 mg/kg, on days 7 and 14 after BCL1 injection. Control mice were injected with the vehicle lipofundin (LPF). There were 15 mice in each group. pV=0.028 comparing survival of ADR treated mice versus ADR+MJ treated mice.

FIG. 5: Combined effects of MJ and 2DG on different cell lines. Cytotoxicity is calculated as % of control untreated cells, mean±s.e. of triplicates. The expected values are assuming additivity. The observed effect values are those actually generated by the combinations of MJ and the cytotoxic drugs. pV<0.05 comparing expected effect of MJ+2DG versus observed effect in CT-26 and D122 cells at all indicated concentrations of MJ (FIGS. 5A and 5B) and at 0.5 mM MJ in MCF cells (FIG. 5C). The difference between expected and observed effect in the case of DA3 (FIG. 5D) cells was not significant.

### DETAILED DESCRIPTION OF THE PRESENT INVENTION

The present invention relates to compositions and methods for treating cancer, by administering a combination comprising a jasmonate derivative in combination with at least one other agent selected from a chemotherapeutic agent and an inhibitor of glycolysis. The jasmonate derivative and the at least one other agent are administered in a collective amount to provide a therapeutic effect, preferably a synergistic effect.

The present invention thus relates to a method for treating cancer in a subject in need thereof, comprising administering to the subject a jasmonate derivative in combination with at least one other agent selected from a chemotherapeutic agent, an inhibitor of glycolysis and combinations thereof, wherein the jasmonate derivative and the at least one other agent together provide a synergistic therapeutic effect.

In another embodiment, the present invention relates to a method for inhibiting cancer cell proliferation, comprising contacting cancer cells with a jasmonate derivative in combination with at least one other agent selected from a chemotherapeutic agent, an inhibitor of glycolysis and combinations thereof, wherein the jasmonate derivative and the at least one other agent together provide a synergistic effect.

In yet another embodiment, the present invention relates to the use of a jasmonate derivative in combination with at least one other agent selected from a chemotherapeutic agent, an inhibitor of glycolysis and combinations thereof, wherein the jasmonate derivative and the at least one other agent together provide a synergistic therapeutic effect.

The combination therapy can provide a therapeutic advantage in view of the differential toxicity associated with the two individual treatments. For example, treatment with jasmonate derivative can lead to a particular toxicity that is not seen with the chemotherapeutic agent or the glycolysis inhibitor, and vice versa. As such, this differential toxicity can permit each treatment to be administered at a dose at which said toxicities do not exist or are minimal, such that together the combination therapy provides a therapeutic dose while avoiding the toxicities of each of the constituents of the combination agents. Furthermore, when the therapeutic effects achieved as a result of the combination treatment are enhanced or synergistic, i.e., significantly better than additive therapeutic effects, the doses of each of the agents can be reduced even further, thus lowering the associated toxicities to an even greater extent.

The terms “synergistic”, “cooperative” and “super-additive” and their various grammatical variations are used interchangeably herein. An interaction between methyl jasmonate and another agent is considered to be synergistic, cooperative or super-additive when the observed effect (e.g., cytotoxicity) in the presence of the drugs together is higher than the sum of the individual effects (e.g., cytotoxicities) of each drug administered separately. In one embodiment, the observed combined effects of the drugs is significantly higher than the sum of the individual effects. The term significant means that the observed p<0.05.

### Jasmonate Derivatives

Any jasmonate derivative, including jasmonic acid, can be used in the combinations of the present invention. As used herein, the term “jasmonate derivative” includes all salts, hydrates, solvates, polymorphs, optical isomers, geometrical isomers, enantiomers, diastereomers, and mixtures thereof of the particular jasmonate derivative.

In a currently preferred embodiment, the jasmonate derivative is methyl jasmonate, which is chemically designated methyl 3-oxo-2-(2-pentenyl)cyclopentaneacetic acid.

In one currently preferred embodiment, the jasmonate derivative is methyl jasmonate. In another currently preferred embodiment, the jasmonate derivative is a compound represented by the formula:

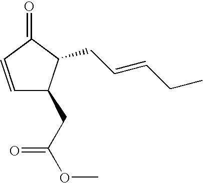

In another currently preferred embodiment, the jasmonate derivative is a compound represented by the formula

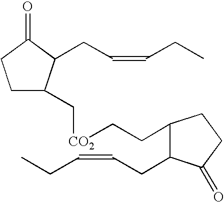

In another currently preferred embodiment, the jasmonate is a compound of formula 9:

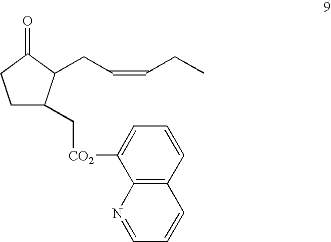

In other embodiments, however, the jasmonate derivative, can be jasmonic acid or any derivative thereof. Suitable jasmonate derivatives include, but are not limited to derivatives described in A) U.S. Pat. No. 6,469,061 and PCT International Patent Application Publication No. WO 02/080890; B) PCT International Patent Application Publication No. WO 2005/054172; C) PCT International Patent Application Publication No. WO 2007/066336; D) PCT International Patent Application Publication No. WO 2007/066337; and E) jasmonate-amino acid conjugate compounds. The contents of each of the aforementioned references are incorporated by reference herein in their entirety as if fully set forth herein.

Non-limiting examples of suitable jasmonate derivatives include:

A) Compounds disclosed in U.S. Pat. No. 6,469,061 and WO 02/080890, represented by the structure of formula I:

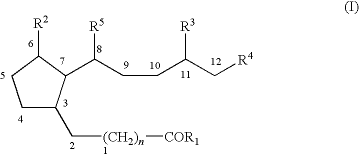

wherein:
n is 0, 1, or 2;
R¹ is OH, alkoxy, O-glucosyl, or imino,
R² is OH, O, alkoxy or O-glucosyl,
R³, R⁴ and R⁵ are H, OH, alkoxy or O-glucosyl, and/or wherein R¹ and R², or R¹ and R⁴ together form a lactone, and further wherein the bonds between C3:C7, C4:C5, and C9:C10 may be double or single bonds; or a derivative of said formula, wherein the derivative has at least one of the following:
a lower acyl side chain at C3 (free acid or ester or conjugate), a keto or hydroxy (free hydroxy or ester) moiety at the C6 carbon, or an n-pentenyl or n-pentyl side chain at C₇;
   and salts, hydrates, solvates, polymorphs, optical isomers, geometrical isomers, enantiomers, diastereomers, and mixtures thereof.

Exemplary jasmonate derivatives include, but are not limited to, methyl jasmonate, jasmonic acid, jasmone, 7-iso-jasmonic acid, 9,10-dihydrojasmonic acid, 2,3-didehydrojasmonic acid, 3,4-didehydrojasmonic acid, 3,7-didehydrojasmonic acid, 4,5-didehydrojasmonic acid, 4,5-didehydro-7-iso-jasmonic acid, cucurbic acid, 6-epi-cucurbic acid, 6-epi-cucurbic-acid-lactone, 12-hydroxy-jasmonic acid, 12-hydroxy-jasmonic-acid-lactone, 11-hydroxy-jasmonic acid, 8-hydroxy-jasmonic acid, homo-jasmonic acid, dihomo-jasmonic acid, 11-hydroxy-dihomo-jasmonic acid, 8-hydroxy-dihomo-jasmonic acid, tuberonic acid, tuberonic acid-O-β-glucopyranoside, cucurbic acid-O-β-glucopyranoside, 5,6-didehydrojasmonic acid, 6,7-didehydro-jasmonic acid, 7,8-didehydrojasmonic acid, cis-jasmone, methyl-dihydro-isojasmonate, dihydro-jasmone, amino acid conjugates of jasmonic acid, the lower alkyl esters of said jasmonic acids.

B) Compounds disclosed in WO 2005/054172, represented by the structure of formula II:

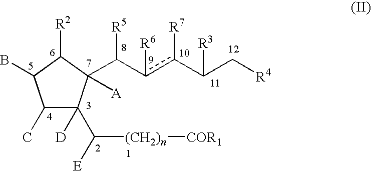

wherein
n is 0, 1, or 2;
R¹ is OH, C₁ to C₁₂ alkoxy, C₁ to C₁₂ substituted alkoxy, aryloxy, O-glucosyl or imino;
R² is OH, C₁ to C₁₂ alkoxy, C₁ to C₁₂ substituted alkoxy, O-glucosyl, oxo, alkyl or imino;
R³, R⁴, R⁵, R⁶, R⁷, A, B, C, D and E are each independently H, halogen, OH, C₁ to C₁₂ alkoxy, C₁ to C₁₂ substituted alkoxy, aryloxy, O-glucosyl, C₁ to C₁₂ alkyl or C₁ to C₁₂ substituted alkyl; wherein R¹ and R², or R¹1 and R⁴ may form together a lactone which is optionally substituted; wherein the bonds between C3:C7, C4: C5, and C9:C10 may independently be double bonds or single bonds;
provided that at least one of R³, R⁴, R⁵, R⁶, R⁷, A, B, C, D and E is a halogen
   and salts, hydrates, solvates, polymorphs, optical isomers, geometrical isomers, enantiomers, diastereomers, and mixtures thereof.

Exemplary jasmonate derivatives include, but are not limited to: methyl jasmonate di-bromide (MJDB), methyl jasmonate tetrabromide (MJTB), a compound wherein R⁶ and R⁷ are each fluoro, a compound wherein R⁶ and R⁷ are each iodo, a compound wherein R⁶ and R⁷ are each chloro, a compound wherein one of R⁶ and R⁷ is iodo and the other is hydroxy, and a compound wherein one of R⁶ and R⁷ is iodo and the other is methoxy.

C) Compounds disclosed in PCT International Patent Application PCT/IL2006/001408, represented by the structure of formula III:

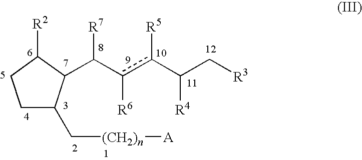

wherein
A is selected from the group consisting of:
   a) COR¹;
   b) O—COR¹⁰; and
   c) OR¹¹;
R¹ is selected from the group consisting of
   a) heteroaryloxy;
   b) —O[(CH₂)ₚO)]ₘ—R¹²;
   c) a group of the formula:

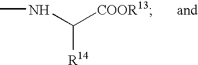

d) when at least one of R³, R⁴, R⁵, R⁶ and R⁷ is haloalkyl or when R⁵ and R⁶, together with the carbons to which they are attached form a C₃-C₈ cycloalkyl or a C₃-C₈ cycloalkyl substituted by halo, R¹ can further represent hydrogen or unsubstituted or substituted C₁-C₁₂ alkyl;
R² is selected from the group consisting of hydrogen, unsubstituted or substituted C₁-C₁₂ alkyl, unsubstituted or substituted C₃-C₈ cycloalkyl, unsubstituted or substituted aryl, unsubstituted or substituted heteroaryl, OR⁸, oxo and NR^{9a}R^{9b};
R³, R⁴, R⁵, R⁶ and R⁷ are each independently selected from the group consisting of hydrogen, halogen, unsubstituted or substituted C₁-C₁₂ alkyl, unsubstituted or substituted C₁-C₁₂ haloalkyl, unsubstituted or substituted C₃-C₉ cycloalkyl, unsubstituted or substituted aryl, unsubstituted or substituted heteroaryl, OR⁸ and NR^{9a}R^{9b},
or R⁵ and R⁶ together with the carbons to which they are attached form a C₃-C₈ cycloalkyl or a C₃-C₈ cycloalkyl substituted by halo;
or one of R⁵ and R⁶ represents an oxygen atom which is bonded to C₆, thereby forming an oxygen-containing 6 or 5 membered heterocyclic ring, respectively;
wherein the bond between C₉ and C₁₀ can be a single or double bond;
R⁸, R^{9a} and R^{9b} are each independently selected from the group consisting of hydrogen, unsubstituted or substituted C₁-C₁₂ alkyl, unsubstituted or substituted C₃-C₈ cycloalkyl, unsubstituted or substituted aryl, unsubstituted or substituted heteroaryl, glucosyl, or R^{9a} and R^{9b} can together with the nitrogen to which they are attached form an unsubstituted or substituted heterocyclic or heteroaromatic ring optionally containing one or more additional heteroatom selected from O, N and S;
R¹⁰ is selected from the group consisting of hydrogen, unsubstituted or substituted C₁-C₁₂ alkyl, unsubstituted or substituted C₃-C₈ cycloalkyl, unsubstituted or substituted aryl and unsubstituted or substituted heteroaryl;
R¹¹ and R¹² are each independently hydrogen or a hydroxy protecting group;
R¹³ is a carboxy protecting group;
R¹⁴ is the residue of a natural or unnatural amino acid;
n is selected from 0, 1 and 2;
m is an integer of 1 to 20; and
p is an integer of 1 to 12;
including salts, hydrates, solvates, polymorphs, optical isomers, geometrical isomers, enantiomers, diastereomers, and mixtures thereof.

Specific examples of the compounds of formula III include, but are not limited to:

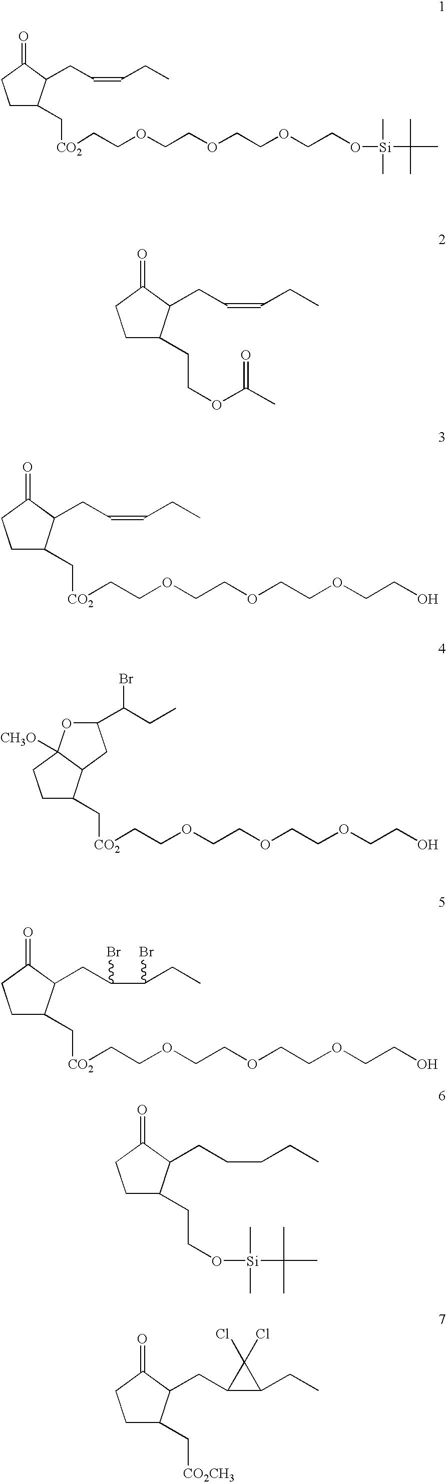

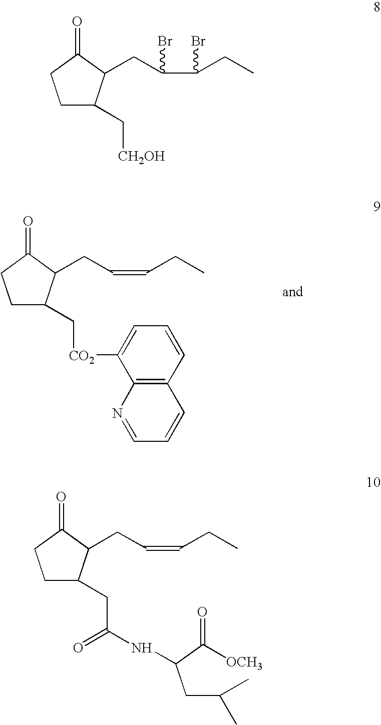

Another example includes a jasmonate derivative represented by the structure of formula 12.

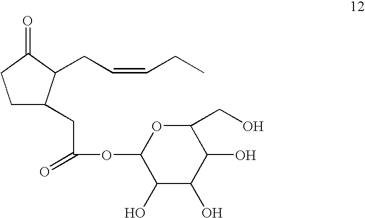

D) Compounds disclosed in PCT International Patent Publication WO 2007/066337, including:
a) Compounds represented by the structure of formula IV:

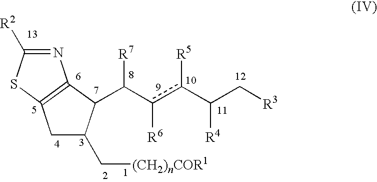

wherein
n is 0, 1, or 2;
R¹ is selected from the group consisting of hydrogen, unsubstituted or substituted C₁-C₁₂ alkyl, unsubstituted or substituted C₃-C₈ cycloalkyl, unsubstituted or substituted aryl, unsubstituted or substituted heteroaryl, a natural or unnatural amino acid, a peptide, OR¹ and NR^{9a}R^{9b};
R² is selected from the group consisting of hydrogen, unsubstituted or substituted C₁-C₁₂ alkyl, unsubstituted or substituted C₃-C₈ cycloalkyl, unsubstituted or substituted aryl, unsubstituted or substituted heteroaryl, OR⁸, NR^{9a}R^{9b}, NHCOR¹⁰ and NHSO₂R¹¹;
R³, R⁴, R⁵, R⁶ and R⁷ are each independently selected from the group consisting of hydrogen, unsubstituted or substituted C₁-C₁₂ alkyl, unsubstituted or substituted C₃-C₈ cycloalkyl, unsubstituted or substituted aryl, unsubstituted or substituted heteroaryl, OR⁸ and NR^{9a}R^{9b};
wherein the bond between C₉ and C₁₀ can be a single or a double bond; and
R⁸, R^{9a}, R^{9b}, R¹⁰ and R¹¹, are each independently selected from the group consisting of hydrogen, unsubstituted or substituted C₁-C₁₂ alkyl, unsubstituted or substituted C₃-C₈ cycloalkyl, unsubstituted or substituted aryl, unsubstituted or substituted heteroaryl, glucosyl, or R^{9a} and R^{9b} can together with the nitrogen to which they are attached form an unsubstituted or substituted heterocyclic or heteroaromatic ring optionally containing one or more additional heteroatom selected from O, N and S;
   including salts, hydrates, solvates, polymorphs, optical isomers, geometrical isomers, enantiomers, diastereomers, and mixtures thereof.

Specific examples of the compounds of formula IV include, but are not limited to:

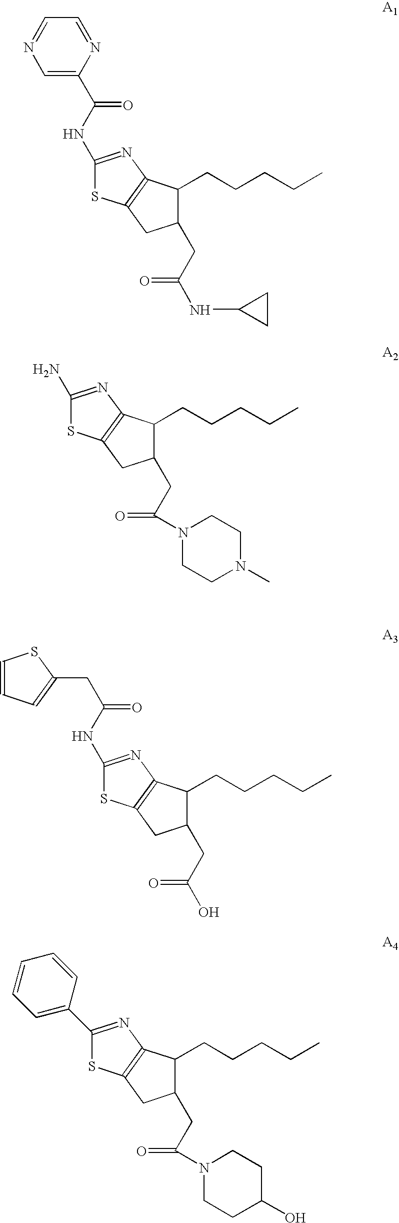

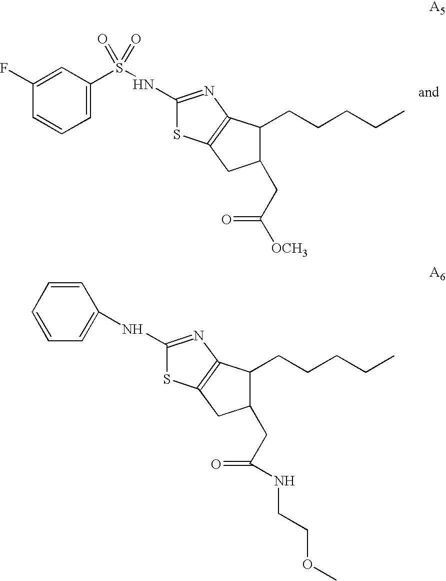

b) Compounds represented by the structure of formula V:

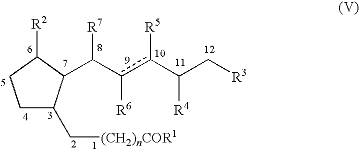

wherein
n is independently at each occurrence 0, 1, or 2;
R¹ is a group of the formula:

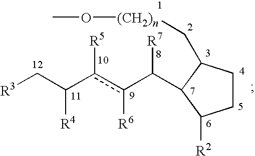

R² is independently at each occurrence selected from the group consisting of hydrogen, unsubstituted or substituted C₁-C₁₂ alkyl, unsubstituted or substituted C₃-C₈ cycloalkyl, unsubstituted or substituted aryl, unsubstituted or substituted heteroaryl, OR⁸, oxo and NR^{9a}R^{9b};
R³, R⁴, R⁵, R⁶ and R⁷ are each independently at each occurrence selected from the group consisting of hydrogen, unsubstituted or substituted C₁-C₁₂ alkyl, unsubstituted or substituted C₃-C₈ cycloalkyl, unsubstituted or substituted aryl, unsubstituted or substituted heteroaryl, OR⁸ and NR^{9a}R^{9b};
wherein the bond between C₉ and C₁₀ can independently at each occurrence be a single or a double bond; and
R⁸, R^{9a} and R^{9b} are each independently at each occurrence selected from the group consisting of hydrogen, unsubstituted or substituted C₁-C₁₂ alkyl, unsubstituted or substituted C₃-C₈ cycloalkyl, unsubstituted or substituted aryl, unsubstituted or substituted heteroaryl, glucosyl, or R^{9a} and R^{9b} can together with the nitrogen to which they are attached form an unsubstituted or substituted heterocyclic or heteroaromatic ring optionally containing one or more additional heteroatom selected from O, N and S;
including salts, hydrates, solvates, polymorphs, optical isomers, geometrical isomers, enantiomers, diastereomers, and mixtures thereof.

A specific example of the compounds of the formula V is:

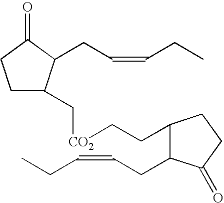

c) Compounds represented by the structure of formula VI:

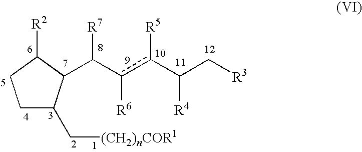

wherein
n is 0, 1, or 2;
R¹ is a natural or unnatural amino acid or a peptide;
R² is selected from the group consisting of hydrogen, unsubstituted or substituted C₁-C₁₂ alkyl, unsubstituted or substituted C₃-C₈ cycloalkyl, unsubstituted or substituted aryl, unsubstituted or substituted heteroaryl, OR⁸, oxo and NR^{9a}R^{9b};
R³, R⁴, R⁵, R⁶ and R⁷ are each independently selected from the group consisting of hydrogen, unsubstituted or substituted C₁-C₁₂ alkyl, unsubstituted or substituted C₃-C₈ cycloalkyl, unsubstituted or substituted aryl, unsubstituted or substituted heteroaryl, OR⁸ and NR^{9a}R^{9b};
wherein the bond between C₉ and C₁₀ can be a single or a double bond; and
R⁸, R^{9a} and R^{9b} are each independently selected from the group consisting of hydrogen, unsubstituted or substituted C₁-C₁₂ alkyl, unsubstituted or substituted C₃-C₈ cycloalkyl, unsubstituted or substituted aryl, unsubstituted or substituted heteroaryl, glucosyl, or R^{9a} and R^{9b} can together with the nitrogen to which they are attached form an unsubstituted or substituted heterocyclic or heteroaromatic ring optionally containing one or more additional heteroatom selected from O, N and S;
   including salts, hydrates, solvates, polymorphs, optical isomers, geometrical isomers, enantiomers, diastereomers, and mixtures thereof.

The amino acid residue in the compounds of formula VI can be a residue of any natural or unnatural amino acid. Currently preferred amino acids are leucine and tryptophan. However, any other natural and unnatural amino acid defined herein and known to a person of skill in the art can be incorporated into the jasmonate-amino acid derivatives of the present invention. Alternatively, the group R¹ can represent a peptide sequence comprising two or more amino acids, which can be natural amino acids, unnatural amino acids, or a combination thereof.

Examples of the compounds of formula VI include, but are not limited to:

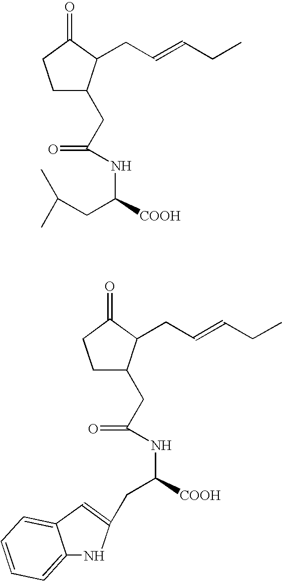

d) Dimeric, oligomeric or polymeric jasmonate derivatives comprising a plurality of covalently linked jasmonic acid moieties Compounds represented by the structure of formula VII:

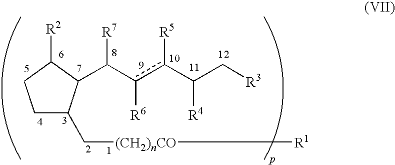

wherein
n is independently at each occurrence 0, 1, or 2;
p is 2, 3, 4, 5 or 6;
R¹ a linker selected from the group consisting of —O—, polyoxy C₁-C₁₂ alkylene and a sugar moiety;
R² is independently at each occurrence selected from the group consisting of hydrogen, unsubstituted or substituted C₁-C₁₂ alkyl, unsubstituted or substituted C₃-C₈ cycloalkyl, unsubstituted or substituted aryl, unsubstituted or substituted heteroaryl, OR⁸, oxo and NR^{9a}R^{9b};
R³, R⁴, R⁵, R⁶ and R⁷ are each independently at each occurrence selected from the group consisting of hydrogen, unsubstituted or substituted C₁-C₁₂ alkyl, unsubstituted or substituted C₃-C₈ cycloalkyl, unsubstituted or substituted aryl, unsubstituted or substituted heteroaryl, OR⁸ and NR^{9a}R^{9b};
wherein the bond between C₉ and C₁₀ can independently at each occurrence be a single or a double bond; and
R⁸, R^{9a} and R^{9b} are each independently at each occurrence selected from the group consisting of hydrogen, unsubstituted or substituted C₁-C₁₂ alkyl, unsubstituted or substituted C₃-C₈ cycloalkyl, unsubstituted or substituted aryl, unsubstituted or substituted heteroaryl, glucosyl, or R^{9a} and R^{9b} can together with the nitrogen to which they are attached form an unsubstituted or substituted heterocyclic or heteroaromatic ring optionally containing one or more additional heteroatom selected from O, N and S;

including salts, hydrates, solvates, polymorphs, optical isomers, geometrical isomers, enantiomers, diastereomers, and mixtures thereof.

Specific examples of the compounds of the formula VII include, but are not limited to:

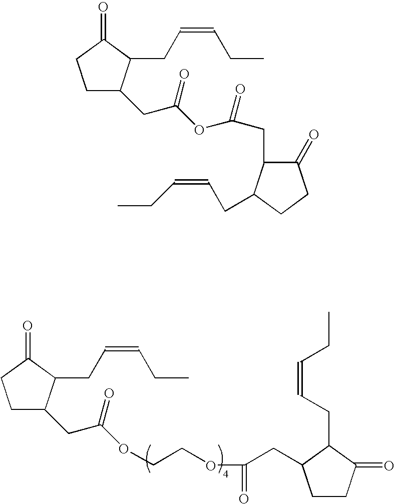

e) Oligomeric compounds comprising a plurality of jasmonate moieties linked via a linker sugar moiety, represented by the structure of formula VIII:

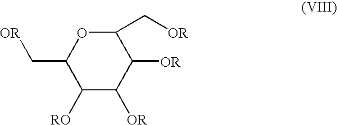

wherein
R is represented by the formula:

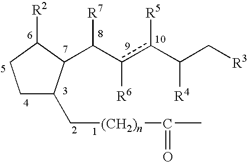

wherein each of R², R³, R⁴, R⁵, R⁶ and R⁷ is as defined above.

A specific example of the compounds of the formula VIII is:

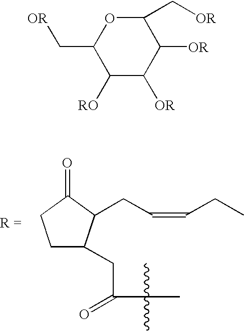

E) Jasmonic Acid Amino-Acid Conjugates:

Jasmonic acids conjugated via the carboxyl group to amino acids occur in nature (Plant Hormones, Davies P J, ed., Kluwer Academic Publishers, London, 2004, pp. 618, 620). Several jasmonic acid-amino acid conjugates have been synthetically prepared. The amino acids include glycine, alanine, valine, leucine and isoleucine. (Jikumaru Y. et al. Biosci. Biotechnol. Biochem. 68, 1461-1466, 2004). The contents of these references are incorporated by reference in their entirety as if fully set forth herein. All of these conjugates can be used in the methods of the present invention.

All stereoisomers of the above jasmonate derivatives are contemplated, either in admixture or in pure or substantially pure form. The jasmonate derivatives can have asymmetric centers at any of the atoms. Consequently, the compounds can exist in enantiomeric or diastereomeric forms or in mixtures thereof. The present invention contemplates the use of any racemates (i.e. mixtures containing equal amounts of each enantiomers), enantiomerically enriched mixtures (i.e., mixtures enriched for one enantiomer), pure enantiomers or diastereomers, or any mixtures thereof. The chiral centers can be designated as R or S or R,S or d,D, l,L or d,l, D,L. Compounds comprising amino acid residues include residues of D-amino acids, L-amino acids, or racemic derivatives of amino acids. Compounds comprising sugar residues include residues of D-sugars, L-sugars, or racemic derivatives of sugars. Residues of D-sugars, which appear in nature, are preferred. In addition, several of the compounds of the invention contain one or more double bonds. The present invention intends to encompass all structural and geometrical isomers including cis, trans, E and Z isomers, independently at each occurrence.

One or more of the compounds of the invention, may be present as a salt. The term “salt” encompasses both basic and acid addition salts, including but not limited to carboxylate salts or salts with amine nitrogens, and include salts formed with the organic and inorganic anions and cations discussed below. Furthermore, the term includes salts that form by standard acid-base reactions with basic groups (such as amino groups) and organic or inorganic acids. Such acids include hydrochloric, hydrofluoric, trifluoroacetic, sulfuric, phosphoric, acetic, succinic, citric, lactic, maleic, fumaric, palmitic, cholic, pamoic, mucic, D-glutamic, D-camphoric, glutaric, phthalic, tartaric, lauric, stearic, salicylic, methanesulfonic, benzenesulfonic, sorbic, picric, benzoic, cinnamic, and like acids.

The term “organic or inorganic cation” refers to counter-ions for the carboxylate anion of a carboxylate salt. The counter-ions are chosen from the alkali and alkaline earth metals, (such as lithium, sodium, potassium, barium, aluminum and calcium); ammonium and mono-, di- and tri-alkyl amines such as trimethylamine, cyclohexylamine; and the organic cations, such as dibenzylammonium, benzylammonium, 2-hydroxyethylammonium, bis(2-hydroxyethyl)ammonium, phenylethylbenzylammonium, dibenzylethylenediammonium, and like cations. See, for example, “Pharmaceutical Salts,” Berge et al., J. Pharm. Sci., 66:1-19 (1977), which is incorporated herein by reference. Other cations encompassed by the above term include the protonated form of procaine, quinine and N-methylglucosamine, and the protonated forms of basic amino acids such as glycine, ornithine, histidine, phenylglycine, lysine and arginine. Furthermore, any zwitterionic form of the instant compounds formed by a carboxylic acid and an amino group are also contemplated.

The present invention also includes solvates of the compounds of the present invention and salts thereof. “Solvate” means a physical association of a compound of the invention with one or more solvent molecules. This physical association involves varying degrees of ionic and covalent bonding, including hydrogen bonding. In certain instances the solvate will be capable of isolation. “Solvate” encompasses both solution-phase and isolatable solvates. Non-limiting examples of suitable solvates include ethanolates, methanolates and the like. “Hydrate” is a solvate wherein the solvent molecule is water.

The present invention also includes polymorphs of the compounds of the present invention and salts thereof. The term “polymorph” refers to a particular crystalline state of a substance, which can be characterized by particular physical properties such as X-ray diffraction, IR spectra, melting point, and the like.

### Chemotherapeutic Agents

Suitable chemotherapeutic agents for use in the combinations of the present invention include, but are not limited to, alkylating agents, antibiotic agents, antimetabolic agents, hormonal agents, plant-derived agents, anti-angiogenic agents, differentiation inducing agents, cell growth arrest inducing agents, apoptosis inducing agents, cytotoxic agents, agents affecting cell bioenergetics, biologic agents, e.g., monoclonal antibodies, kinase inhibitors and inhibitors of growth factors and their receptors, gene therapy agents, cell therapy, e.g., stem cells, or any combination thereof.

Alkylating agents are drugs which impair cell function by forming covalent bonds with amino, carboxyl, suflhydryl and phosphate groups in biologically important molecules. The most important sites of alkylation are DNA, RNA and proteins. Alkylating agents depend on cell proliferation for activity but are not cell-cycle-phase-specific. Alkylating agents suitable for use in the present invention include, but are not limited to, bischloroethylamines (nitrogen mustards, e.g. chlorambucil, cyclophosphamide, ifosfamide, mechlorethamine, melphalan, uracil mustard), aziridines (e.g. thiotepa), alkyl alkone sulfonates (e.g. busulfan), nitroso-ureas (e.g. BCNU, carmustine, lomustine, streptozocin), nonclassic alkylating agents (e.g., altretamine, dacarbazine, and procarbazine), and platinum compounds (e.g., carboplastin and cisplatin).

Antitumor antibiotics like adriamycin intercalate DNA at guanine-cytosine and guanine-thymine sequences, resulting in spontaneous oxidation and formation of free oxygen radicals that cause strand breakage (7). Other antibiotic agents suitable for use in the present invention include, but are not limited to, anthracyclines (e.g. doxorubicin, daunorubicin, epirubicin, idarubicin and anthracenedione), mitomycin C, bleomycin, dactinomycin, and plicatomycin.

Antimetabolic agents suitable for use in the present invention include but are not limited to, floxuridine, fluorouracil, methotrexate, leucovorin, hydroxyurea, thioguanine, mercaptopurine, cytarabine, pentostatin, fludarabine phosphate, cladribine, asparaginase, and gemcitabine.

Hormonal agents suitable for use in the present invention, include but are not limited to, an estrogen, a progestogen, an antiesterogen, an androgen, an antiandrogen, an LHRH analogue, an aromatase inhibitor, diethylstibestrol, tamoxifen, toremifene, fluoxymesterol, raloxifene, bicalutamide, nilutamide, flutamide, aminoglutethimide, tetrazole, ketoconazole, goserelin acetate, leuprolide, megestrol acetate, and mifepristone.

Plant derived agents include taxanes, which are semisynthetic derivatives of extracted precursors from the needles of yew plants. These drugs have a novel 14-member ring, the taxane. Unlike the vinca alkaloids, which cause microtubular disassembly, the taxanes (e.g., taxol) promote microtubular assembly and stability, therefore blocking the cell cycle in mitosis (7). Other plant derived agents include, but are not limited to, vincristine, vinblastine, vindesine, vinzolidine, vinorelbine, etoposide, teniposide, and docetaxel.

Biologic agents suitable for use in the present invention include, but are not limited to immuno-modulating proteins, monoclonal antibodies against tumor antigens, tumor suppressor genes, kinase inhibitors and inhibitors of growth factors and their receptors and cancer vaccines. For example, the immuno-modulating protein can be interleukin 2, interleukin 4, interleukin 12, interferon El interferon D, interferon alpha, erythropoietin, granulocyte-CSF, granulocyte, macrophage-CSF, bacillus Calmette-Guerin, levamisole, or octreotide. Furthermore, the tumor suppressor gene can be DPC-4, NF-1, NF-2, RB, p53, WT1, BRCA, or BRCA2.

Agents affecting cell bioenergetics affecting cellular ATP levels and/or molecules/activities regulating these levels

Recent developments have introduced, in addition to the traditional cytotoxic and hormonal therapies, additional therapies for the treatment of cancer. For example, many forms of gene therapy are undergoing preclinical or clinical trials. In addition, approaches are currently under development, that are based on the inhibition of tumor vascularization (angiogenesis). The aim of this concept is to cut off the tumor from nutrition and oxygen supply provided by a newly built tumor vascular system. In addition, cancer therapy is also being attempted by the induction of terminal differentiation of the neoplastic cells. Suitable differentiation agents include hydroxamic acids, derivatives of vitamin D and retinoic acid, steroid hormones, growth factors, tumor promoters, and inhibitors of DNA or RNA synthesis. Also, histone deacetylase inhibitors are suitable chemotherapeutic agent to be used in the present invention.

In currently preferred embodiments, the chemotherapeutic agent is a nitroso-urea (e.g., 1,3-bis[2-chloroethyl]-10-nitroso-urea (BCNU), a platinum compound (e.g., cisplatin), a taxane derivative (e.g., taxol or its derivatives), an antitumor antibiotic (e.g., adriamycin), or any combination thereof.

### Inhibitors of Glycolysis

As described above, it has recently been shown that cells under hypoxic conditions are more sensitive than cells under aerobic conditions to agents that inhibit glycolysis, such as 2-deoxy-D-glucose (2DG). It has been postulated that combining such agents with chemotherapeutic drugs, which target the rapidly dividing aerobic cells, should raise the overall efficacies of these treatments. It has been shown that the combination of 2DG and cisplatin is more effective than either agent alone when applied to various cell lines that are rapidly proliferating in vitro. Similar in vitro synergism has been observed with the combination of 2DG and adriamycin, and it has furthermore been shown that 2DG significantly enhances the cytotoxic effects of anticancer agents like topoisomerase inhibitors (etoposide and camptothecin) and an antibiotic drug (bleomycin) in established human tumor cell lines.

Therefore, in one embodiment, the present invention contemplates the use of a jasmonate derivative in combination with a glycolytic inhibitor such as 2DG, optionally further in combination with one or more additional chemotherapeutic agents described above.

Other inhibitors of glycolysis include oxamate and its derivatives. See, for example, Hamilton E, Fennell M, Stafford D M. Acta Oncol. 1995; 34(3):429-33, the contents of which are incorporated by reference in their entirety.

### Mechanism of Action and Therapeutic Use

The present invention relates to a method for treating cancer in a subject in need thereof, comprising administering to the subject a jasmonate derivative in combination with at least one other agent selected from a chemotherapeutic agent, an inhibitor of glycolysis and combinations thereof, wherein the jasmonate derivative and the at least one other agent together provide a therapeutic effect.

In another embodiment, the present invention relates to a method for treating cancer in a subject in need thereof, comprising administering to the subject a jasmonate derivative in combination with at least one other agent selected from a chemotherapeutic agent, an inhibitor of glycolysis and combinations thereof, wherein the jasmonate derivative and the at least one other agent together provide a synergistic therapeutic effect.

In another embodiment, the present invention relates to the use of a combination comprising a jasmonate derivative and at least one other agent selected from a chemotherapeutic agent, an inhibitor of glycolysis, for the preparation of a medicament for the treatment of cancer, wherein the first and the second amounts together provide a synergistic therapeutic effect.

As demonstrated, herein, cooperative effects between MJ and several anti-cancer drugs were observed in six cell lines arising from different major types of malignancies: breast, lung, prostate and pancreatic carcinomas as well as leukemia. Furthermore, MJ significantly enhanced the anti-leukemic effect of adriamycin in vivo. Four different chemotherapeutic drugs in routine clinical usage were evaluated. These were chosen based on their mechanism of action which differs from that of MJ. Without wishing to be bound by any particular mechanism or theory, it is contemplated that drugs with different mechanisms of action are promising combinations for cancer therapy. Nevertheless, the cytotoxic effect of each of these drugs is mediated, though indirectly, via mitochondrial perturbation. Thus, the mitochondria serve as a central point of cellular life and death decisions. Without wishing to be bound by any particular mechanism or theory, it is proposed that combinations of drugs that affect these organelles, though through different specific mechanisms, could merge to yield super-additive efficacy. This results in IC50 values of the various chemotherapeutic drugs being drastically lowered in the presence of MJ, pointing towards the potential of reducing unwanted side effects.

MJ has previously been shown to act against leukemic cells from CLL patients while sparing normal lymphocytes (1, 2, 3). Thus, MJ could enhance currently-available therapy against CLL without causing side effects. Moreover, it has previously been shown that MJ can kill p53-mutant cells (6) and cell expressing high levels of P-gp. Consequently, a drug combination including MJ should potentially have an advantage in treating patients exhibiting drug resistance.

In addition, a combination of MJ and the glycolysis inhibitor 2DG exhibited a super-additive cytotoxic effect on various cancer cells. Without wishing to be bound by any particular mechanism or theory, it is proposed that this reflects cooperation between the inhibition of both oxidative phosphorylation and glycolysis as the two major cellular sources of ATP biosynthesis.

The term “cancer” in the context of the present invention includes all types of neoplasm whether in the form of solid or non-solid tumors, from all origins, and includes both malignant and premalignant conditions as well as their metastasis. The combinations of the present invention are active against a wide range of cancers. The combinations of the present invention are active against a wide range of cancers, including carcinomas, sarcomas, myelomas, leukemias, lymphomas and mixed type tumors. Particular categories of tumors amenable to treatment include lymphoproliferative disorders, breast cancer, ovarian cancer, prostate cancer, cervical cancer, endometrial cancer, bone cancer, liver cancer, stomach cancer, colon cancer, pancreatic cancer, cancer of the thyroid, head and neck cancer, cancer of the central nervous system, cancer of the peripheral nervous system, skin cancer, kidney cancer, as well as metastases of all the above. Particular types of tumors amenable to treatment include: hepatocellular carcinoma, hematoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, Ewing's tumor, leiomyosarcoma, rhabdotheliosarcoma, invasive ductal carcinoma, papillary adenocarcinoma, melanoma, squamous cell carcinoma, basal cell carcinoma, adenocarcinoma (well differentiated, moderately differentiated, poorly differentiated or undifferentiated), renal cell carcinoma, hypernephroma, hypernephroid adenocarcinoma, bile duct carcinoma, choriocarcinoma, seminoma, embryonal carcinoma, Wilms' tumor, testicular tumor, lung carcinoma including small cell, non-small and large cell lung carcinoma, bladder carcinoma, glioma, astrocyoma, medulloblastoma, craniopharyngioma, ependymoma, pinealoma, retinoblastoma, neuroblastoma, colon carcinoma, rectal carcinoma, hematopoietic malignancies including all types of leukemia and lymphoma including: acute myelogenous leukemia, acute myelocytic leukemia, acute lymphocytic leukemia, chronic myelogenous leukemia, chronic lymphocytic leukemia, mast cell leukemia, multiple myeloma, myeloid lymphoma, Hodgkin's lymphoma, non-Hodgkin's lymphoma.

In particular, the combinations of the present invention are active against breast cancer, kidney cancer, stomach cancer, leukemia, including lymphoblastic leukemia, lung carcinoma, melanoma and colon cancer. In one embodiment, the subject is a mammal, preferably a human. However, the present invention also contemplates using the compounds of the present invention for non-mammal humans, e.g., in veterinary medicine.

It is to be understood that whenever the terms “treating or inhibiting cancer” or “treating or inhibiting a malignant (cancer) cell proliferation” are used herein in the description and in the claims, they are intended to encompass tumor formation, primary tumors, tumor progression or tumor metastasis.

The term “inhibition of proliferation” in relation to cancer cells, in the context of the present invention refers to a decrease in at least one of the following: number of cells (due to cell death which may be necrotic, apoptotic or any other type of cell death or combinations thereof) as compared to control; decrease in growth rates of cells, i.e. the total number of cells may increase but at a lower level or at a lower rate than the increase in control; decrease in the invasiveness of cells (as determined for example by soft agar assay) as compared to control even if their total number has not changed; progression from a less differentiated cell type to a more differentiated cell type; a deceleration in the neoplastic transformation; or alternatively the slowing of the progression of the cancer cells from one stage to the next.

The term “treatment of cancer” in the context of the present invention includes at least one of the following: a decrease in the rate of growth of the cancer (i.e. the cancer still grows but at a slower rate); cessation of growth of the cancerous growth, i.e., stasis of the tumor growth, and, in preferred cases, the tumor diminishes or is reduced in size. The term also includes reduction in the number of metastases, reduction in the number of new metastases formed, slowing of the progression of cancer from one stage to the other and a decrease in the angiogenesis induced by the cancer. In most preferred cases, the tumor is totally eliminated. Additionally included in this term is lengthening of the survival period of the subject undergoing treatment, lengthening the time of diseases progression, tumor regression, and the like. This term also encompasses prevention for prophylactic situations or for those individuals who are susceptible to contracting a tumor. The administration of the compounds of the present invention will reduce the likelihood of the individual contracting the disease. In preferred situations, the individual to whom the compound is administered does not contract the disease.

As used herein, the term “administering” refers to bringing in contact with a compound of the present invention. Administration can be accomplished to cells or tissue cultures, or to living organisms, for example humans. In one embodiment, the present invention encompasses administering the compounds of the present invention to a human subject.

A “therapeutic” treatment is a treatment administered to a subject who exhibits signs of pathology for the purpose of diminishing or eliminating those signs. A “therapeutically effective amount” is that amount of compound which is sufficient to provide a beneficial effect to the subject to which the compound is administered. A “synergistic therapeutically effective amount” means that the combination treatment regimen produces a significantly better anticancer result (e.g., cell growth arrest, apoptosis, induction of differentiation, cell death) than the additive effects of each constituent when it is administered alone at a therapeutic dose. Standard statistical analysis can be employed to determine when the results are significantly better. For example, a Mann-Whitney Test or some other generally accepted statistical analysis can be employed.

### Pharmaceutical Compositions

Although the combinations of the present invention can be administered alone, it is contemplated that the components of the combination will be administered in pharmaceutical compositions further containing at least one pharmaceutically acceptable carrier or excipient. Each of the components can be administered in a separate pharmaceutical composition, or the combination can be administered in one pharmaceutical composition.

Thus, in one embodiment, the present invention also contemplates pharmaceutical compositions that include a first amount of a jasmonate derivative in combination with a second amount of at least one other agent selected from a chemotherapeutic agent and an inhibitor of glycolysis. The first and the second amounts together provide a therapeutic anti-cancer effect which is, in one embodiment, synergistic.

In another embodiment, the present invention contemplates a first pharmaceutical composition that includes a first amount of a jasmonate derivative and a second pharmaceutical composition that includes a second amount of at least one other agent selected from a chemotherapeutic agent and an inhibitor of glycolysis. The first and the second amounts together provide a therapeutic anti-cancer effect which is, in one embodiment, synergistic. If the combination comprises more than two components, then the total amount of jasmonate derivative, chemotherapeutic agent and/or inhibitor of glycolysis provide a therapeutic anti-cancer effect which is, in one embodiment, synergistic.

The pharmaceutical compositions of the present invention can be formulated for administration by a variety of routes including oral, rectal, transdermal, parenteral (subcutaneous, intraperitoneal, intravenous, intra-arterial, transdermal and intramuscular), topical, intranasal, or via a suppository. Such compositions are prepared in a manner well known in the pharmaceutical art and comprise as an active ingredient at least one compound of the present invention as described hereinabove, and a pharmaceutically acceptable excipient or a carrier. The term “pharmaceutically acceptable” means approved by a regulatory agency of the Federal or a state government or listed in the U.S. Pharmacopeia or other generally recognized pharmacopeia for use in animals and, more particularly, in humans.

During the preparation of the pharmaceutical compositions according to the present invention the active ingredient is usually mixed with a carrier or excipient, which may be a solid, semi-solid, or liquid material. The compositions can be in the form of tablets, pills, capsules, pellets, granules, powders, lozenges, sachets, cachets, elixirs, suspensions, dispersions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

The carriers may be any of those conventionally used and are limited only by chemical-physical considerations, such as solubility and lack of reactivity with the compound of the invention, and by the route of administration. The choice of carrier will be determined by the particular method used to administer the pharmaceutical composition. Some examples of suitable carriers include lactose, glucose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water and methylcellulose. The formulations can additionally include lubricating agents such as talc, magnesium stearate, and mineral oil; wetting agents, surfactants, emulsifying and suspending agents; preserving agents such as methyl- and propylhydroxybenzoates; sweetening agents; flavoring agents, colorants, buffering agents (e.g., acetates, citrates or phosphates), disintegrating agents, moistening agents, antibacterial agents, antioxidants (e.g., ascorbic acid or sodium bisulfite), chelating agents (e.g., ethylenediaminetetraacetic acid), and agents for the adjustment of tonicity such as sodium chloride. Other pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerin, propylene glycol or other synthetic solvents. Water is a preferred carrier when the pharmaceutical composition is administered intravenously. Saline solutions and aqueous dextrose and glycerol solutions can also be employed as liquid carriers, particularly for injectable solutions.

In one embodiment, in the pharmaceutical composition the active ingredient is dissolved in any acceptable lipid carrier (e.g., fatty acids, oils to form, for example, a micelle or a liposome).

For preparing solid compositions such as tablets, the principal active ingredient(s) is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, from about 0.1 mg to about 2000 mg, from about 0.1 mg to about 500 mg, from about 1 mg to about 100 mg, from about 100 mg to about 250 mg, etc. of the active ingredient(s) of the present invention.

Any method can be used to prepare the pharmaceutical compositions. Solid dosage forms can be prepared by wet granulation, dry granulation, direct compression and the like. The solid dosage forms of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer, which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compositions of the present invention may be incorporated, for administration orally or by injection, include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insulation include solutions and suspensions in pharmaceutically acceptable aqueous or organic solvents, or mixtures thereof, and powders. The liquid or solid compositions may contain suitable pharmaceutically acceptable excipients as described above. Preferably the compositions are administered by the oral or nasal respiratory route for local or systemic effect. Compositions in preferably pharmaceutically acceptable solvents may be nebulized by use of inert gases. Nebulized solutions may be breathed directly from the nebulizing device or the nebulizing device may be attached to a face masks tent, or intermittent positive pressure breathing machine. Solution, suspension, or powder compositions may be administered, preferably orally or nasally, from devices that deliver the formulation in an appropriate manner.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art.

In yet another embodiment, the composition is prepared for topical administration, e.g. as an ointment, a gel a drop or a cream. For topical administration to body surfaces using, for example, creams, gels, drops, ointments and the like, the compounds of the present invention can be prepared and applied in a physiologically acceptable diluent with or without a pharmaceutical carrier. The present invention may be used topically or transdermally to treat cancer, for example, melanoma. Adjuvants for topical or gel base forms may include, for example, sodium carboxymethylcellulose, polyacrylates, polyoxyethylene-polyoxypropylene-block polymers, polyethylene glycol and wood wax alcohols.

Alternative formulations include nasal sprays, liposomal formulations, slow-release formulations, pumps delivering the drugs into the body (including mechanical or osmotic pumps) controlled-release formulations and the like, as are known in the art.

The compositions are preferably formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

In preparing a formulation, it may be necessary to mill the active ingredient to provide the appropriate particle size prior to combining with the other ingredients. If the active compound is substantially insoluble, it ordinarily is milled to a particle size of less than 200 mesh. If the active ingredient is substantially water soluble, the particle size is normally adjusted by milling to provide a substantially uniform distribution in the formulation, e.g. about 40 mesh.

It may be desirable to administer the pharmaceutical composition of the invention locally to the area in need of treatment; this may be achieved by, for example, and not by way of limitation, local infusion during surgery, infusion to the liver via feeding blood vessels with or without surgery, topical application, e.g., in conjunction with a wound dressing after surgery, by injection, by means of a catheter, by means of a suppository, or by means of an implant, said implant being of a porous, non-porous, or gelatinous material. According to some preferred embodiments, administration can be by direct injection e.g., via a syringe, at the site of a tumor or neoplastic or pre-neoplastic tissue.

The compounds may also be administered by any convenient route, for example by infusion or bolus injection, by absorption through epithelial linings (e.g., oral mucosa, rectal and intestinal mucosa, etc.), and may be administered together with other therapeutically active agents. It is preferred that administration is localized, but it may be systemic. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir. Pulmonary administration can also be employed, e.g., by use of an inhaler or nebulizer, and formulation with an aerosolizing agent.

A compound of the present invention can be delivered in an immediate release or in a controlled release system. In one embodiment, an infusion pump may be used to administer a compound of the invention, such as one that is used for delivering chemotherapy to specific organs or tumors (see Buchwald et al., 1980, Surgery 88: 507; Saudek et al., 1989, N. Engl. J. Med. 321: 574). In a preferred form, a compound of the invention is administered in combination with a biodegradable, biocompatible polymeric implant, which releases the compound over a controlled period of time at a selected site. Examples of preferred polymeric materials include polyanhydrides, polyorthoesters, polyglycolic acid, polylactic acid, polyethylene vinyl acetate, copolymers and blends thereof (See, Medical applications of controlled release, Langer and Wise (eds.), 1974, CRC Pres., Boca Raton, Fla.). In yet another embodiment, a controlled release system can be placed in proximity of the therapeutic target, thus requiring only a fraction of the systemic dose.

Furthermore, at times, the pharmaceutical compositions may be formulated for parenteral administration (subcutaneous, intravenous, intraarterial, transdermal, intraperitoneal or intramuscular injection) and may include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that include suspending agents, solubilizers, thickening agents, stabilizers, and preservatives. Oils such as petroleum, animal, vegetable, or synthetic oils and soaps such as fatty alkali metal, ammonium, and triethanolamine salts, and suitable detergents may also be used for parenteral administration. The above formulations may also be used for direct intra-tumoral injection. Further, in order to minimize or eliminate irritation at the site of injection, the compositions may contain one or more nonionic surfactants. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described and known in the art.

Alternatively, the combinations of the present invention can be used in hemodialysis such as leukophoresis and other related methods, e.g., blood is drawn from the patient by a variety of methods such as dialysis through a column/hollow fiber membrane, cartridge etc, is treated with the jasmonate derivatives and/or chemotherapeutic drug and or glycolysis inhibitor Ex-vivo, and returned to the patient following treatment. Such treatment methods are well known and described in the art. See, e.g., Kolho et al. (J. Med. Virol. 1993, 40(4): 318-21); Ting et al. (Transplantation, 1978, 25(1): 31-3); the contents of which are hereby incorporated by reference in their entirety.

### Doses and Dosing Schedules

The treatment with the jasmonate derivative and the at least other chemotherapeutic agent and/or anti-glycolysis inhibitor can take place sequentially in any order, simultaneously or a combination thereof. For example, administration of a jasmonate derivative can take place prior to, after or at the same time as administration of the chemotherapeutic agent and/or the inhibitor of glycolysis. For example, a total treatment period can be decided for the jasmonate derivative. The additional agent(s) (chemotherapeutic agent and/or the inhibitor of glycolysis) can be administered prior to onset of treatment with the jasmonate derivative or following treatment with the jasmonate derivative. In addition, the additional agent(s) can be administered during the period of jasmonate derivative administration but does not need to occur over the entire jasmonate derivative treatment period. In another embodiment, the treatment regimen includes pre-treatment with one agent, either the jasmonate derivative or the chemotherapeutic agent/glycolysis inhibitor, followed by the addition of the other agent or agents. Alternating sequences of administration are also contemplated. Alternating administration includes administration of a jasmonate derivative, a chemotherapeutic agent and/or glycolysis inhibitor in alternating sequences, e.g., jasmonate derivative, followed by chemotherapeutic agent, followed by glycolysis inhibitor, followed by jasmonate derivative, etc.

The amount of a compound of the invention (i.e., jasmonate derivative/chemotherapeutic agent/inhibitor of glycolysis) that will be effective in the treatment of a particular disorder or condition, including cancer, will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. A preferred dosage will be within the range of 0.01-1000 mg/kg of body weight, 0.1 mg/kg to 100 mg/kg, 1 mg/kg to 100 mg/kg, 10 mg/kg to 75 mg/kg, 0.1-1 mg/kg, etc. Exemplary (non-limiting) amounts of the jasmonate derivative/chemotherapeutic agent/inhibitor of glycolysis include 0.1 mg/kg, 0.2 mg/kg, 0.5 mg/kg, 1 mg/kg, 5 mg/kg, 10 mg/kg, 20 mg/kg, 50 mg/kg, 60 mg/kg, 75 mg/kg and 100 mg/kg. Alternatively, the amount administered can be measured and expressed as molarity of the administered compound. By way of illustration and not limitation, a jasmonate derivative (e.g., methyl jasmonate) can be administered in a range of 0.1-10 mM, e.g., 0.1, 0.25, 0.5, 1 and 2 mM. Alternatively, the amount administered can be measured and expressed as mg/ml, μg/ml, or ng/ml. By way of illustration and not limitation, a chemotherapeutic agent can be administered in an amount of 1 ng/ml to 100 mg/ml, for example 1-1000 ng/ml, 1-100 ng/ml, 1-1000 μg/ml, 1-100 μg/ml, 1-1000 mg/ml, 1-100 mg/ml, etc. Effective doses may be extrapolated from dose-response curves derived from in vitro or animal model test bioassays or systems. When a synergistic effect is observed, the overall dose of each of the components may be lower, thus the side effects experienced by the subject may be significantly lower, while a sufficient chemotherapeutic effect is nevertheless achieved. As demonstrated in the Experimental section herein, the jasmonate derivative methyl jasmonate, in combination with various chemotherapeutic agents (adriamycin, taxol, BCNU and cisplatin) exhibit synergistic anti-proliferative effects at various concentration ranges, in-vitro and in-vivo.

In one embodiment, the combination therapy reduces the amount of each of its component by a factor of 2, i.e., each component is given at half the dose as compared with single agent therapy, and still achieves the same or similar therapeutic effect. in another embodiment, the combination therapy reduces the amount of each of its component by a factor of 5, 10, 20, 50 or 100. As demonstrated herein, the IC50 of chemotherapeutic agents as anti-proliferative agents in various cancer cells are reduced as compared to the IC50 of the chemotherapeutic agent, when administered alone.

The administration schedule will depend on several factors such as the cancer being treated, the severity and progression, the patient population, age, weight etc. For example, the compositions of the invention can be taken once-daily, twice-daily, thrice daily, once-weekly or once-monthly. In addition, the administration can be continuous, i.e., every day, or intermittently. The terms “intermittent” or “intermittently” as used herein means stopping and starting at either regular or irregular intervals. For example, intermittent administration can be administration one to six days per week or it may mean administration in cycles (e.g. daily administration for two to eight consecutive weeks, then a rest period with no administration for up to one week) or it may mean administration on alternate days. The different components of the combination can, independently of the other, follow different dosing schedules.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXPERIMENTAL DETAILS SECTION

### Example 1

### Materials and Methods

### Chemicals:

Methyl Jasmonate [methyl 3-oxo-2-(2-pentenyl)cyclopentaneacetic acid], 2-Deoxy-D-glucose (2DG), 1,3-bis{2-Chloroethyl}-1-nitroso-urea (BCNU) and cis Diammineplatinum (II) dichloride (cisplatin) were purchased from Sigma-Aldrich Chemie GmbH, Steinheim, Germany. Adriamycin was purchased from Pharmacia Italia S.p.A. and Taxol from MeadJohnson, USA. Methyl Jasmonate was dissolved in absolute ethanol to give a stock solution of 500 mM. Further dilutions of MJ and dilutions of the cytotoxic drugs were performed in culture medium. The final concentration of ethanol in cultures did not exceed 0.6%. For in vivo experiments, adriamycin was dissolved in Phosphate Buffer Saline.

### Tumor Cell Lines:

CT26 is a murine colon carcinoma. DA-3 is a murine mammary adenocarcinoma. TRAMP C1 is a murine prostate adenocarcinoma. MCF7 is a human breast adenocarcinoma. M IA PaCa-2 is a human pancreatic carcinoma. D122 is a murine lung carcinoma. BCL1 is a murine B cell leukemia. All cell lines were purchased from ATCC (Rockville, Md., USA), except for DA3 and D122 which were kindly provided by Prof. Y. Keisari (Tel-Aviv University, Israel).

The cell lines were found negative for mycoplasma infection as revealed by VenorGem mycoplasma detection kit 25T (Minerva Biolabs, Berlin, Germany).

The cells were maintained in a humidified atmosphere, at 37° C. with 5% CO₂. CT26 and DA-3 cells were maintained in Dulbecco's modified Eagle's medium (Biological Industries, Beit-Haemek, Israel), supplemented with 10% FCS, 2 mM L-glutamine, 100 U ml⁻¹ penicillin, 100 μg ml⁻¹ streptomycin, 1 mM sodium pyruvate and 1:100 dilution of nonessential amino acids (all purchased from Biological Industries, Israel).

MCF7, MIA PaCa-2 and BCL1 cells were maintained in RPMI-1640 medium (Biological Industries, Israel) supplemented with 10% FCS, 2 mM L-glutamine, 100 U ml⁻¹ penicillin and 100 μg ml⁻¹ streptomycin.

TRAMP C1 cells were grown in Dulbecco's modified Eagle's medium supplemented with 10% FCS, 2 mM L-glutamine, 100 U ml⁻¹ penicillin, 100 μg ml⁻¹ streptomycin, 1 mM sodium pyruvate, 1:100 dilution of nonessential amino acids, 5 μg/ml bovine insulin and 10 nM dehydroisoandrosterone.

BCL1 cells, which are unable to grow continuously in culture, were maintained in BALB/c mice. Blood was taken from the tail vein of BCL1-bearing mice on day 23-28 post inoculation, and depleted of red blood cells (RBC) using RBC lysis buffer (Sigma-Aldrich). The purified leukemic cells were used for in vitro and in vivo experiments.

### Cytotoxicity Assay:

All cells (except for BCL1) were plated into 96-well microtiter plates (Corning) at a density of 2*10³ cells per well and were allowed to adhere prior to treatment. BCL1 cells were seeded at a density of 20*10⁴ cells per well. The cells were exposed to MJ, cytotoxic drugs, 2DG or their combinations at different concentrations for 24 hours. Cytotoxic drugs and 2DG were added 1 h prior to MJ addition.

Inhibition of cell proliferation was determined by the CellTiter 96 Aqueous Non-Radioactive Cell Proliferation Assay (Promega, Madison, Wis., USA). Upon completion of a given experiment, 20 μl of a mixture (20:1) of MTS (a tetrazolium compound, at a final concentration of 333 μg/ml)+phenazine methosulfate (at a final concentration of 25 μM) were added to each well of the 96-well plate for 1 h at 37° C. This allowed for the development of the reaction in which dehydrogenases reduce the MTS in metabolically active cells. Since the cells were not washed before the addition of MTS, there were no problems observed with potentially loosely adherent or non-adherent cells. Soluble MTS formazan product at wavelength 490 nm was measured with the CERES 900 HDi ELISA reader (Bio-Tek Instruments, Highland Park, Vt., USA). Optical density is directly proportional to the number of living cells in culture. Cytotoxicity (%) was calculated in the following way: [(absorbance of control cells−absorbance of drug-treated cells)/absorbance of control cells]×100.

### In Vivo Study:

Balb/c male mice (7-8 weeks old) were obtained from the breeding colony of Tel-Aviv University, Israel. Animal care and experimentation were carried out in accordance with Tel-Aviv University guidelines and approved by the institutional animal use and care committee. Mice were kept in cages under standard food and housing conditions during the experiments.

2×10⁴ BCL1 cells, freshly extracted from BCL1 leukemia bearing BALB/c mice, were inoculated intraperitoneally (i.p.) into mice in 100 μl PBS to produce tumor growth. Methyl jasmonate at 60 mg/kg was administered to animals 5 times a week, daily, for 4 weeks, starting one day after cell inoculation. This dose of MJ was found to be well tolerated by animals in preliminary experiments. MJ was dissolved in a lipid formulation, Lipofundin (B. Braun Melsungen, Melsungen, Germany). The adriamycin was given to mice twice, on days 7 and 14, at 4 mg/kg, i.p. Control mice were treated with the vehicle alone. The survival of mice was monitored daily.

### Statistical Analysis

Statistical significance in in vitro experiments was assessed using the two-tailed Student's t-test. P<0.05 was considered statistically significant. The survival curves (Kaplan-Meier test) and statistical analysis (Mantel-Cox test) were carried out using Statistical software.

### Example 2

### Cytotoxic Effect of MJ Towards Tumor Cell Lines In Vitro

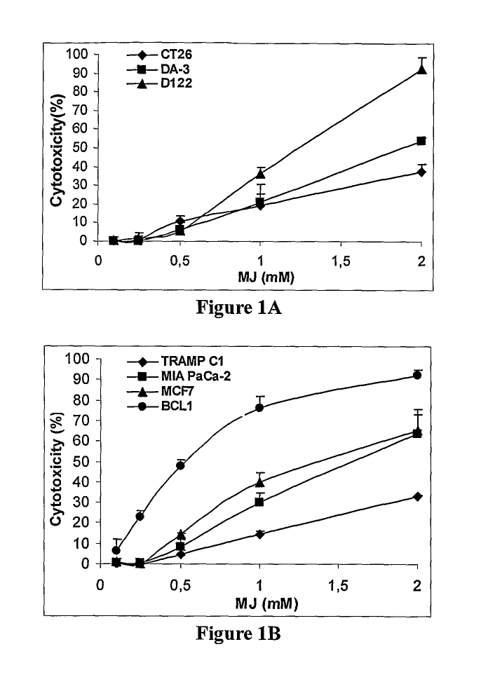

The cytotoxic activity of MJ was tested in vitro against 6 adherent cell lines and 1 ex vivo mouse cell line. Each cell line was exposed to MJ for 24 h at concentrations ranging from 0.1 mM to 2 mM and cytotoxicity was determined as described in Methods. The IC50 values are summarized in Table 1. As can be seen from FIG. 1, MJ exerted cytotoxic effects at concentrations at or above 0.25 mM. All cell lines responded in a dose-dependent fashion to MJ.

TABLE 1
IC50 of MJ in different cell lines

| IC50 values | MJ (mM) |
|---|---|
| D122 | 1.22 ± 0.06 |
| DA-3 | 1.91 ± 0.08 |
| CT26 | 2.59 ± 0.12 |
| TRAMP C1 | 2.94 ± 0.13 |
| MIA PaCA-2 | 1.46 ± 0.13 |
| MCF7 | 1.49 ± 0.06 |
| BCL1 | 0.56 ± 0.09 |

### Example 3

### Cytotoxic Effect of Combined Treatment with MJ and Chemotherapeutic Drugs on Carcinoma Cell Lines In Vitro

The cooperative effect of MJ with traditional chemotherapeutic drugs was investigated. Anticancer agents are rarely used as monotherapies. Effective chemotherapy usually depends on the proper and effective combination of two or more agents. Four drugs with different modes of action were selected. BCNU, cisplatin, taxol and adriamycin were assessed for cooperativity in combination with a fixed concentration of MJ in 7 cell lines. The MJ concentration was chosen in accordance with dose response data (FIG. 1) such that the cytotoxicity of MJ didn't exceed 40%. The interaction between MJ and another agent was considered cooperative (super additive) when the difference between cytotoxicity in the presence of both drugs together and the sum of the cytotoxicities of each drug administered separately (expected additivity on the graph), yielded a pV<0.05. The summary of these experiments is presented in table 2. As can be seen, MJ does not exhibit cooperative activity with any of the 4 drugs in CT26 cells, while in other cell lines a cooperative effect of MJ was observed with one or two chemotherapeutic drugs.

TABLE 2
Summary of experiments evaluating combinations between MJ and various
chemotherapeutic drugs.

| | | | | MIA | | TRAMP | |
|---|---|---|---|---|---|---|---|
| | DA-3 | D122 | CT26 | PaCA-2 | MCF7 | C1 | BCL1 |
| Cisplatin | − | − | − | + | − | + | − |
| Adria- | − | − | − | − | − | − | + |
| mycin | | | | | | | |
| BCNU | − | − | − | + | − | − | + |
| Taxol | + | + | − | − | + | + | − |

+ combinations yielding, at least at some concentrations, cooperative effect

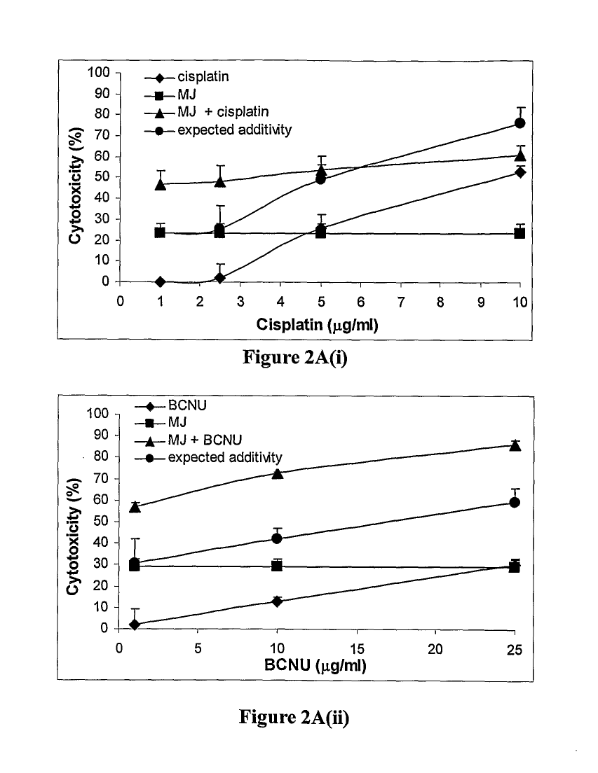

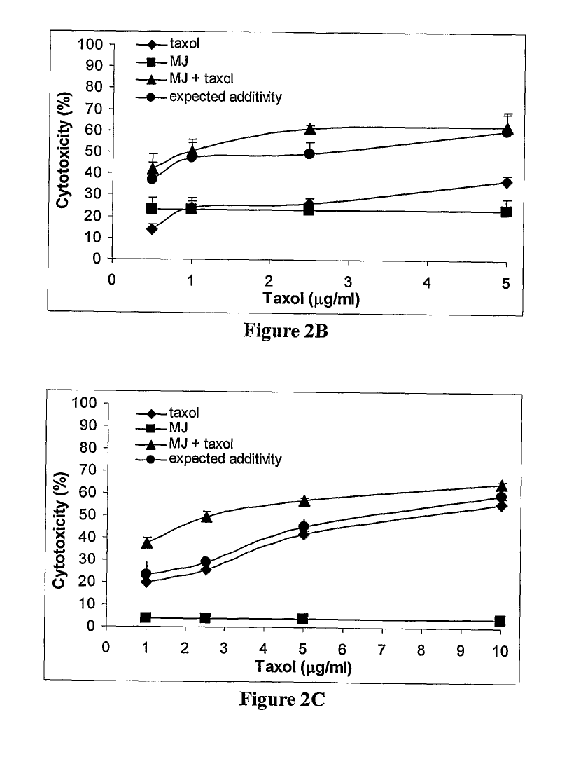

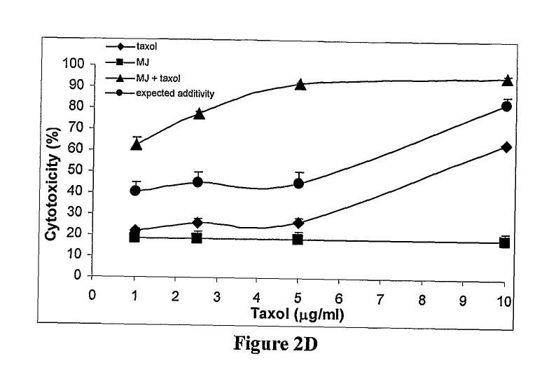

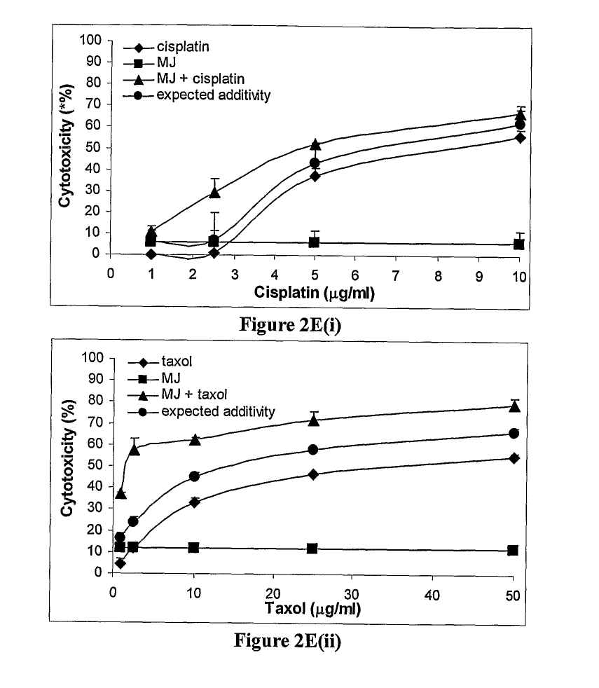

As shown in FIG. 2A, MIA PaCa-2 cells exhibit strong cooperative effects with BCNU at all concentrations tested (1-25 μg/ml), whereas cooperation with cisplatin is exhibited at low cisplatin concentrations (1 and 2.5 μg/ml). The IC50 of BCNU by itself is above 25 μg/ml while that of the combination is less than 1 μg/ml.

In MCF7 cells (FIG. 2B) MJ enhances the cytotoxic capability of taxol when combined with 2.5 μg/ml taxol, whereas the combinations at other concentrations are additive.

In DA-3 cells (FIG. 2C), a cooperative effect of MJ with taxol at 1, 2.5 and 5 μg/ml is observed, while at 10 μg/ml the effect is additive. The IC50 of taxol in this combination is reduced to 2.5 μg/ml whereas the IC50 of taxol alone is 9 μg/ml. Very strong cooperation of taxol and MJ can be seen in D122 cells (FIG. 2D) at all indicated concentrations. The IC50 of taxol alone in this experimental system is 8.2 μg/ml, but it is reduced to less than 1 μg/ml in the presence of MJ.

Cooperation of taxol with MJ is shown in FIG. 2E in TRAMP C1 cells: at all concentrations tested (1-50 μg/ml) the combined effect in TRAMP C1 cells is significantly higher than the expected additivity. The IC 50 of taxol for TRAMP C1 cells is 38 μg/ml. This value is diminished by combined treatment with MJ to 2 μg/ml. Cisplatin also exhibited cooperation with taxol in TRAMP C1 cells at concentrations of 1 and 2.5 μg/ml.

### Example 4

### Cytotoxic Effect of MJ Towards BCL1 Cells In Vitro

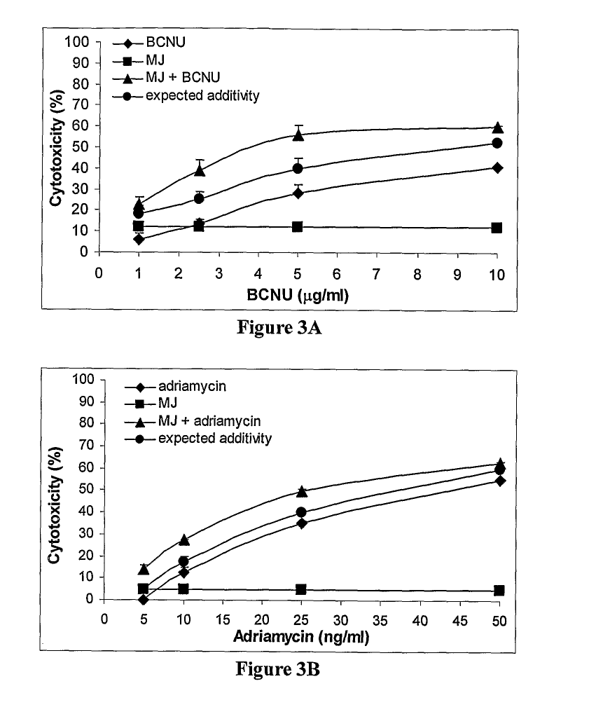

The cytotoxicity of MJ towards BCL1 cells which were freshly extracted from BCL1 leukemia-bearing mice was examined (FIG. 1). These cells were found to be most sensitive to MJ (IC50=0.56). Furthermore, BCL1 cells are considered as a model of human B cell leukemia and MJ killed effectively leukemic cells freshly-drawn from the blood of CLL patients (2,3). Consequently, the possible cooperative effect of MJ with chemotherapeutic drugs in these primary tumor cells was evaluated. The chosen MJ concentration in these experiments (0.1 mM) was much lower than in experiments with carcinoma cells due to the high sensitivity of BCL1 cells. No cooperative effect of MJ with cisplatin and taxol was observed. However, as can be seen in FIG. 3 and as is summarized in Table 2, there is cooperation between MJ and BCNU at 2.5 and 5 μg/ml (pV<0.05) and adriamycin at 10 and 25 ng/ml. At other concentrations an additive effect was observed.

### Example 5

### Combination of MJ and Adriamycin is Synergistic Against BCL1 In Vivo

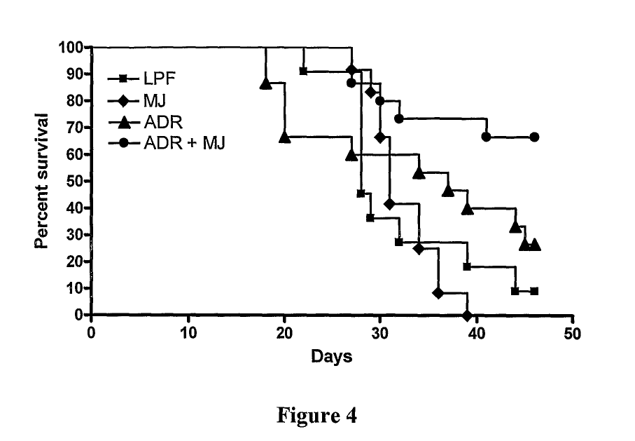

Since adriamycin is used for treatment of leukemia, it was chosen for an experiment assessing a combination with MJ in vivo. BALB/c mice were injected i.p. with 10⁴ freshly extracted BCL1 cells and treated with combination of adriamycin and MJ for 4 weeks. The dose of adriamycin was chosen based on previous in vivo experiments, i.e., at a non-toxic level that exhibits a minimal curing effect. The treatment with MJ started one day after BCL1 injection. MJ was administered every day at 60 mg/kg by intravenous injection, whereas adriamycin was injected twice: on day 7 and on day 14. As can be seen in FIG. 4, the survival of mice treated with MJ+ADR is significantly prolonged (pV=0.028) versus mice treated by MJ or ADR alone. Thus, cooperation between MJ and ADR can be observed not only in vitro, but also in vivo.

### Example 6

### Cytotoxic Effects of Combined Treatment with MJ and 2DG on Different Cell Lines In Vitro

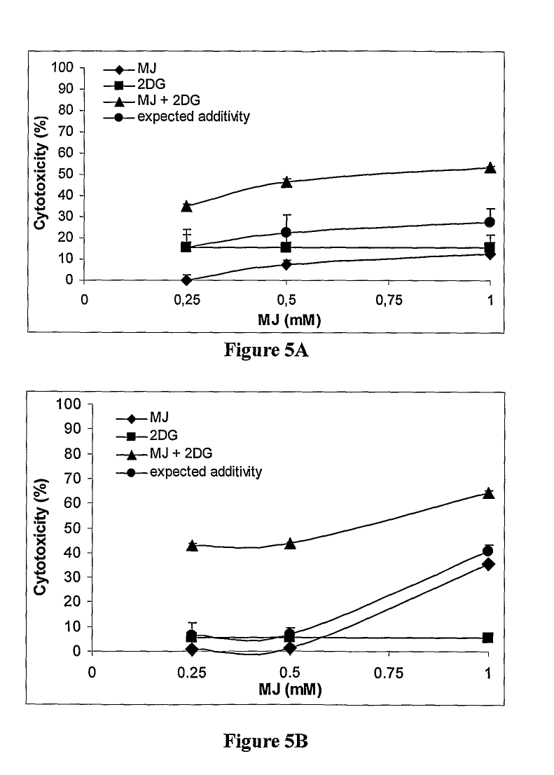

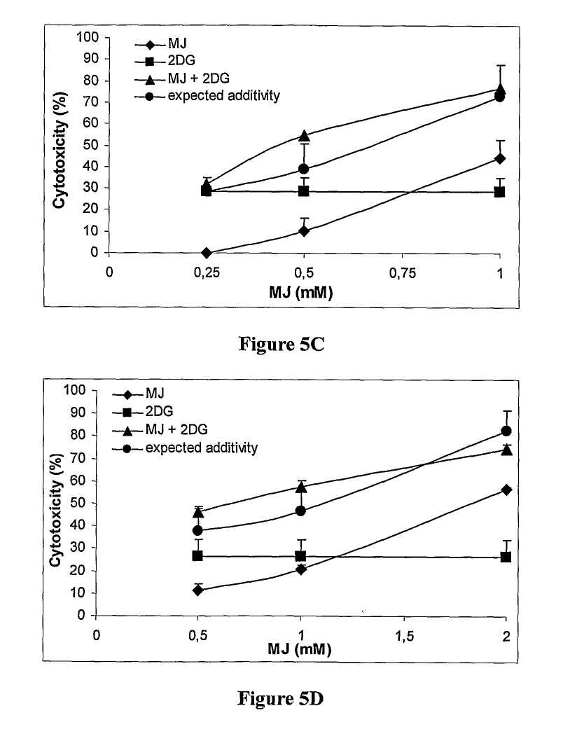

The applicants of the present invention have recently shown that inhibition of glycolysis by 2DG in 29M4.1 B lymphoma cells enhanced the effect of MJ on ATP levels, yielding a drastic depletion of cellular ATP levels which was significantly stronger than the effect caused by MJ alone (6). Therefore, the possible combined effect of MJ with 2DG on cell viability was examined. For this purpose, four cells lines were exposed to different concentrations of MJ with a constant concentration of 2DG. The results of this experiment are summarized in FIG. 5. As shown, 2 DG at 0.5 mM significantly enhanced the cytotoxicity of MJ in CT26 and D122 cells at each of the MJ concentrations (pV<0.05), whereas in MCF7 cells the effect was significant only at 0.5 mM MJ and additive at other concentrations. No cooperative effect was observed in DA-3 cells.

In conclusion, the mitochondriotoxic anti-cancer agent MJ can cooperate with various common chemotherapeutic drugs, as well as a glycolysis inhibitor, both in vitro and in vivo. These data constitute a foundation for the potential clinical use of MJ in drug combinations, possibly also against drug-resistant tumors.

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

### REFERENCES

1. O. Fingrut and E. Flescher. Plant stress hormones suppress the proliferation and induce apoptosis in human cancer cells. Leukemia, 2002; 16:608-616
2. R. Rotem, A. Heyfets, O. Fingrut, D. Blickstein, M. Shaklai, and E. Flescher. Jasmonates: novel anticancer agents acting directly and selectively on human cancer cell mitochondria. Cancer Res, 2005; 65:1984-1993.
3. Flescher E. Jasmonates—a new family of anti-cancer agents. Anticancer Drugs, 2005; 16:911-6.
4. Gold D., Pankova-Kholmyansky I., Fingrut O., Flescher E. The antiparasitic actions of plant jasmonates. J Parasitol, 2003; 89:1242-1244.
5. R. Rotem, O. Fingrut, J. Moskovitz and E. Flescher. The anticancer agent methyl jasmonate induces activation of stress-regulated c-Jun N-terminal kinase and p38 protein kinase in human lymphoid cells. Leukemia, 2003; 17:2230-2234.
6. O. Fingrut, D. Reischer, R. Rotem, N. Goldin, I. Altboum, I. Zan-Bar & E Flescher. Jasmonates induce nonapoptotic death in high-resistance mutant p53-expressing B-lymphoma cells. Br J of Pharmacol, 2005; 146:800-808.
7. Richard Pazdur, Lawrence R. Coia, William J. Hoskins, Lawrence D. Wagman Cancer management: a multidisciplinary approach. Chapter 3: Principles of oncologic pharmacotherapy. 9th Edition, 2005, pp.: 23-42
8. E. Frei and J. P Eder. Principles of Dose, Schedule, and Combination Therapy. Cancer medicine, 2003; chapter 11, pp. 817-837
9. C. P. Reynolds and B. J. Maurer. Evaluating response to antineoplastic drug combinations in tissue culture models. Methods in molecular medicine, 2005; 110: 173-183
10. Hu Y. P., Morales C., Savaraj N., Priebe W., Lampidis T. J. Rho (0) Tumor cells: a model for studying whether mitochondria are targets for rhodamine 123, doxorubicin and other drugs. Biochem. Pharmacol., 2000; 60:1897-1905
11. Liu H., Hu Y. P., Savaraj N., Priebe W., Lampidis T. J. Hypersensitization of tumor cells to glycolytic inhibitors. Biochemistry, 2001; 40:5542-5547
12. Liu H., Hu Y. P., Savaraj N., Priebe W., Lampidis T. J. Hypoxia increases tumor cell sensitivity to glycolytic inhibitors, a strategy for solid tumor therapy (Model C). Biochem. Pharmacol., 2002; 64:1746-1751
13. Yamada M., Tomida A., Yun J., Cai B., Yoshikawa H., Taketani Y., Tsuruo T. Cellular sensitization to cisplatin and carboplatin with decreased removal of platinum-DNA adduct by glucose-regulated stress. Cancer Chemother Pharmacol, 1999; 44: 59-64
14. Kaplan O., Navon G., Lyon R. C., Faustino P. J., Straka E. J., Cohen J. S. Effects of 2-deoxyglucose on drug-sensitive and drug-resistant human breast cancer cells: toxicity and magnetic resonance spectroscopy studies of metabolism. Cancer Res, 1990; 50:544-551
15. Slavin, S, and Strober, S. Spontaneous murine B-cell leukemia. Nature, 1978; 272:624-626
16. Slavin S, Weiss L, Morecki S, Bassat H B, Leizerowitz R, Gamliel H, Korkesh A, Voss R, Polliack A. Ultrastructural, cell membrane, and cytogenetic characteristics of B-cell leukemia, a murine model of chronic lymphocytic leukemia. Cancer Res., 1981; 41:4162-4166
17. Cao T M, Coutre S E. Management of advanced chronic lymphocytic leukemia. Curr Hematol Rep, 2003; 2:65-72
18. Escobar-Diaz E, Lopez-Martin E M, Hernandez del Cerro M, Puig-Kroger A, Soto-Cerrato V, Montaner B, Giralt E, Garcia-Marco J A, Perez-Tomas R, Garcia-Pardo A. AT514, a cyclic depsipeptide from Serratia marcescens, induces apoptosis of B-chronic lymphocytic leukemia cells: interference with the Akt/NF-kappaB survival pathway. Leukemia, 2005; 19:572-579
19. Morra E, Nosari A, Montillo M. Infectious complications in chronic lymphocytic leukemia. Hematol Cell Ther, 1999; 41:145-151
20. Schiavone E M, De Simone M, Palmieri S, Annunziata M, Pocali B, Copia C, D'Amico M R, Vecchio L D, Ferrara F. Fludarabine plus cyclophosphamide for the treatment of advanced chronic lymphocytic leukemia. Eur J Haematol, 2003; 71:23-28

1-34. (canceled)

35. A method for treating cancer or inhibiting cancer cell proliferation in a subject in need thereof, comprising administering to the subject a jasmonate derivative in combination with at least one other agent selected from the group consisting of a nitroso-urea, a platinum compound, a taxane derivative, an antitumor antibiotic and an inhibitor of glycolysis, wherein the jasmonate derivative and the at least one other agent together provide a synergistic therapeutic effect.

36. The method of claim 35 wherein the inhibition of cancer cell proliferation comprises contacting a cancer cell with a jasmonate derivative in combination with at least one other agent.

37. The method of claim 35, wherein the jasmonate derivative is methyl jasmonate.

38. The method of claim 35, wherein the jasmonate derivative is a compound represented by any of formulae:

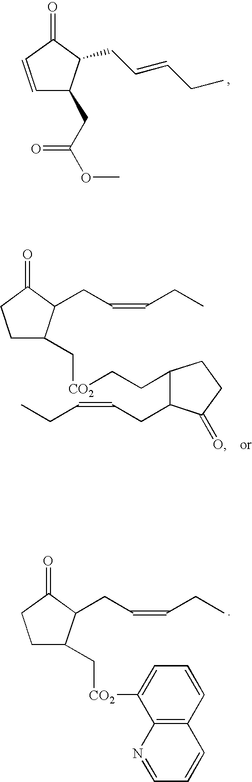

39. The method of claim 35, wherein the nitroso-urea is 1,3-bis[2-chloroethyl]-10-nitroso-urea (BCNU).

40. The method of claim 35, wherein the platinum compound is cisplatin.

41. The method of claim 35, wherein the taxane derivative is taxol.

42. The method of claim 35, wherein the antitumor antibiotic is adriamycin.

43. The method of claim 35, wherein the inhibitor of glycolysis is 2-deoxy-D-glucose (2DG).

44. The method of claim 37, wherein the at least one other agent is selected from the group consisting of 1,3-bis[2-chloroethyl]-10-nitroso-urea (BCNU), cisplatin, taxol and adriamycin.

45. The method of claim 37, wherein the inhibitor of glycolysis is 2-deoxy-D-glucose (2DG).

46. The method of claim 35, wherein the subject is a human.

47. The method of claim 35, wherein the jasmonate derivative and the at least one other agent are administered in the same pharmaceutical composition or in separate pharmaceutical compositions, simultaneously or sequentially, in any order.

48. The method of claim 35, wherein the cancer is a carcinoma selected from breast carcinoma, lung carcinoma, colon carcinoma, prostate carcinoma and pancreatic carcinoma; a sarcoma or leukemia.

49. The method of claim 35, wherein the cancer is selected from a mammary adenocarcinoma, lung carcinoma, and breast adenocarcinoma, the jasmonate derivative is methyl jasmonate, and the at least one other agent is taxol.

50. The method of claim 35, wherein the cancer is pancreatic carcinoma, the jasmonate derivative is methyl jasmonate, and the at least one other agent is cisplatin or BCNU.

51. The method of claim 35, wherein the cancer is prostate adenocarcinoma, the jasmonate derivative is methyl jasmonate, and the at least one other agent is cisplatin or taxol.

52. The method of claim 35, wherein the cancer is B-cell leukemia, the jasmonate derivative is methyl jasmonate, and the at least one other agent is adriamycin or BCNU.

53. The method of claim 35, wherein the cancer is colon carcinoma, lung carcinoma or breast adenocarcinoma, the jasmonate derivative is methyl jasmonate, and the at least one other agent is 2DG.

54. A pharmaceutical composition comprising a jasmonate derivative in combination with at least one other agent selected from the group consisting of a nitroso-urea, a platinum compound, a taxane derivative, an antitumor antibiotic and an inhibitor of glycolysis, wherein the collective amount of jasmonate derivative and the at least one other agent provides a synergistic therapeutic anti-cancer effect.

55. The pharmaceutical composition of claim 54, wherein the composition is in a form suitable for oral administration, intravenous administration by injection, topical administration, administration by inhalation, or administration via a suppository.

56. A method for treating cancer or inhibiting cancer cell proliferation in a subject in need thereof, comprising administering to the subject a jasmonate derivative in combination with at least one other agent selected from a chemotherapeutic agent and an inhibitor of glycolysis, wherein the jasmonate derivative and the at least one other agent together provide a synergistic therapeutic effect.

57. The method of claim 56, wherein the chemotherapeutic agent is selected from the group consisting of an alkylating agent, an antibiotic agent, an antimetabolic agent, a hormonal agent, a plant-derived agent, an anti-angiogenic agent, a differentiation inducing agent, a cell growth arrest inducing agent, an apoptosis inducing agent, a cytotoxic agent, a biologic agent, a gene therapy agent, and any combination thereof.

58. The method of claim 56, wherein the jasmonate derivative is methyl jasmonate.
